(19) United States
(12) Patent Application Publication
(10) Pub. No.: US 2022/0235419 A1
(43) Pub. Date: Jul. 28, 2022

(54) Risk Evaluation of Genomic Susceptibility to Opioid Addiction

(71) Applicants: Genemarkers, LLC, Kalamazoo, MI (US); Ferris State University, Big Rapids, MI (US)

(72) Inventors: Anna Langerveld, Kalamazoo, MI (US); David Bright, Rockford, MI (US); Minji Sohn, Big Rapids, MI (US); Claire Saadeh, DeWitt, MI (US); Susa DeVuyst-Miller, Grand Rapids, MI (US)

(21) Appl. No.: 17/616,222

(22) PCT Filed: Jun. 3, 2020
(86) PCT No.: PCT/US2020/035913

§ 371 Date: Dec. 3, 2021

Related U.S. Application Data
(60) Provisional application No. 62/856,812, filed on Jun. 4, 2019.

(51) Int. Cl. C12Q 1/6883; G16H 50/30; G16H 10/40; G16H 20/70

(52) U.S. Cl. CPC C12Q 1/6883; G16H 50/30; C12Q 1/6827; G16H 20/70; G16H 10/40

(57) ABSTRACT

The present disclosure relates to methods for assessing whether a subject is genetically predisposed to the risk of opioid addiction including opioid relapse or opioid use disorder. The method comprises: (1) performing an allelic analysis on the biological sample to determine the presence of a plurality of pre-determined alleles; (2) assigning a count for each of the alleles in the plurality of pre-determined alleles that was present in the biological sample; (3) determining a risk score based upon a total count, wherein the risk score identifies a severity of the genetic predisposition to opioid addiction or relapse risk; and (4) administering a medical assisted treatment procedure based on the risk score identified in the subject.

### CROSS REFERENCE TO RELATED APPLICATIONS

This application claims priority under 35 U.S.C. § 119(e) to U.S. Provisional Patent Application 62/856,812, filed Jun. 4, 2019. The entire contents of the aforementioned application are hereby incorporated by reference in its entirety, including drawings.

### TECHNICAL FIELD

The present disclosure relates to methods of treating, assessing or preventing opioid use disorder (OUD), and more specifically, obtaining and utilizing a risk score for assessing a genetic predisposition to opioid addiction or opioid addiction relapse in a subject using a plurality of pre-determined alleles.

### BACKGROUND

There is a growing opioid problem in the United States. This national epidemic has been recognized by the Federal government, with pledged support and requests to develop precision medicine based solutions. Prescription drug abuse has led to health problems, addiction, and death. In the United States, 44 people die every day from an overdose of prescription painkillers, more than cocaine and heroin combined.

In the United States, opioid overdose deaths increased by 265% among men and 400% among women between 1999 and 2010. There has been a consistent increase in the prevalence of opioid use disorder (OUD), resulting in medical complications (i.e., nonfatal overdoses), falls and fractures, drug-drug interactions and neonatal conditions. These complications result in costly, preventable expenditures and a great amount of emotional suffering. The opioid epidemic impacts people of all ages, from infants and children to the elderly.

Accordingly, there is a need for techniques able to address and assess risk of opioid addiction and opioid deaths in the United States and across the world.

### SUMMARY

In some aspects, the present disclosure provides a method of assessing whether a subject is at risk of opioid addiction, the method comprising:

(1) determining the presence of a plurality of pre-determined alleles in a biological sample from the subject by assigning a plurality of counts, wherein the plurality of pre-determined alleles comprise two or more genomic targets selected from Table 1;

(2) determining a risk score based upon summing the plurality of counts; and

(3) comparing the risk score with one or more predetermined reference values, wherein the subject is determined to have a risk level of opioid addiction if the risk score is greater than a threshold value using a SNP model.

In other embodiments, the methods comprise obtaining and utilizing an opioid use disorder (OUD) risk score for assessing a genetic predisposition to opioid addiction, the method comprising:

(1) obtaining a biological sample from a subject;

(2) performing an allelic analysis on the biological sample to determine the presence of a plurality of pre-determined alleles, wherein the plurality of pre-determined alleles comprise two or more genomic targets selected from Table 1;

(3) assigning a count for each of the alleles in the plurality of pre-determined alleles that was present in the biological sample;

(4) determining a risk score based upon a total count using a SNP Model, wherein the risk score identifies a severity of the genetic predisposition to opioid addiction; and

(5) administering a medical assisted treatment procedure based on the risk score identified in the subject.

In further embodiments, the methods comprise obtaining and utilizing a relapse risk score for assessing a genetic predisposition to addiction relapse, the method comprising:

(1) obtaining a biological sample from a subject;

(2) performing an allelic analysis on the biological sample to determine the presence of a plurality of pre-determined alleles, wherein the plurality of pre-determined alleles comprise two or more genomic targets selected from Table 1;

(3) assigning a count for each of the alleles in the plurality of pre-determined alleles that was present in the biological sample;

(4) determining a risk score based upon a total count using a SNP Model, wherein the risk score identifies a severity of the genetic predisposition to addiction relapse; and

(5) administering a medical assisted treatment procedure based on the risk score identified in the subject.

In other embodiments, the methods include assessing whether a subject is at risk of opioid addiction, the method comprising:

(1) determining the presence of a plurality of pre-determined alleles in a biological sample from the subject by assigning a plurality of counts, wherein the plurality of pre-determined alleles comprises two or more genomic targets selected in Table 1;

(2) determining a risk score based upon summing the plurality of counts;

(3) comparing the risk score with a predetermined reference value using a SNP Model, wherein the subject is determined to be at high risk of opioid addiction if the risk score is greater than a threshold value as compared to those subjects where the risk score is lower than the threshold value; and

(4) administering a medical assisted treatment procedure based on the risk score identified in the subject.

In still other embodiments, the methods include obtaining and utilizing a relapse risk score for assessing a genetic predisposition to addiction relapse, the method comprising:

(1) obtaining a biological sample from a subject;

(2) performing an allelic analysis on the biological sample to determine the presence of a plurality of pre-determined alleles by assigning a plurality of counts, wherein the plurality of pre-determined alleles comprise one or more genomic targets selected from Table 1;

(3) determining a risk score based upon summing the plurality of counts; and

(4) comparing the risk score with one or more predetermined reference values, wherein the subject is determined to have a risk level of opioid addiction if the risk score is greater than a threshold value using a SNP model.

In some embodiments, the SNP model comprises a sex-stratified single count SNP model, a sex-stratified double count SNP model, a non-sex-stratified single count SNP model, or any combinations thereof.

In some embodiments, the medical assisted treatment procedure comprises monitoring the subject, prescribing an increase dose, prescribing a decreased dose, transitioning the subject from inpatient to outpatient rehabilitation, transitioning the subject from outpatient to inpatient rehabilitation, or any combinations thereof.

In some embodiments, the plurality of pre-determined alleles comprise at least one allele selected from the group consisting of:

allele C+ of gene BDNFOS/antiBDNF (rs11030096);

allele A+ of gene DRD2 (rs1079596);

allele G+ of gene DRD2 (rs1125394);

allele C+ of gene DRD3 (rs9288993);

allele T/T of gene GABRB3 (rs4906902);

allele C/C of gene OPRM1 (rs510769);

allele T/T of gene TACR1 (rs735668);

allele T/T of gene ZNF804A (rs7597593);

allele C+ of gene DRD3 (rs2654754); and/or allele A/A of gene OPRM1 (rs1799971).

In other embodiments, the plurality of pre-determined alleles comprise at least one allele selected from the group consisting of:

allele A/A of gene CNR1 (rs2023239);

allele G+ of gene TACR3 (rs4530637);

allele C+ of gene TACR3 (rs1384401);

allele T/T of gene EXOC4 (rs718656);

allele T+ of gene DRD3 (rs324029);

allele G+ of gene DRD3 (rs6280);

allele G/G of gene CNR1 (rs6928499);

allele G/G of gene CYPB6 (rs3745274); and/or

allele C/C of gene CYP2D6 (rs1065852).

In further embodiments, the plurality of pre-determined alleles comprise at least one allele selected from the group consisting of:

allele C/C of gene CNIH3 (rs1369846);

allele A/A of gene CNIH3 (rs1436171);

allele A/A of gene GRIN3A (rs17189632);

allele C+ of gene HTR3B (rs11606194);

allele C/C of gene OPRD1 (rs2234918);

allele G/G of gene WLS (rs1036066);

allele G+ of gene intergenic (rs965972);

allele C/C of gene MTHFR (rs1801133); and/or

allele G/G of gene MTHFR (rs1801133).

In some embodiments, the plurality of pre-determined alleles comprise at least one allele selected from the group consisting of:

allele T/T of gene DRD3 (rs9825563);

allele T/T of gene GAL (rs948854);

allele C+ of gene NR4A2 (rs1405735);

allele A+ of gene OPRM (rs9479757); and/or

allele T+(A+) of gene CYP3A4 (rs35599367).

In some embodiments, the plurality of pre-determined alleles comprise at least one allele selected from the group consisting of:

chr11:113399438 of gene ANKK1;

chr11:27643996 of gene BDNFOS/antiBDNF;

chr1:224706393 of gene CNIH3;

chr6:88150763 of gene CNR1;

chr16:3745362 of gene CREBBP;

chr22:38287631 of gene CSNK1E;

chr11:113425897 of gene DRD2;

chr11:113441417 of gene DRD2;

chr11:113426463 of gene DRD2;

chr11:113414814 of gene DRD2;

chr11:113412966 of gene DRD2;

chr11:113425564 of gene DRD2;

chr3:114162776 of gene DRD3;

chr3:114140326 of gene DRD3;

chr11:636784 of gene DRD4;

chr15:26774621 of gene GABRB3;

chr19:1005231 of gene GABRB3;

chr1:163535374 of gene intergenic g.163535374G;

chr1:28855013 of gene OPRD1;

chr1:28863085 of gene OPRD1;

chr6:154040884 of gene OPRM1;

chr8:56447926 of gene PENK;

chr5:1446274 of gene SLC6A3;

chr2:75198602 of gene TACR1;

chr2:75135918 of gene TACR1;

chr4:103643921 of gene TACR3;

chr4:103585232 of gene TACR3;

chr1:68194522 of gene WLS;

chr2:184668853 of gene ZNF804A; and/or

chr2:184913701 of gene ZNF804A.

In some embodiments, the plurality of pre-determined alleles further comprise at least one allele selected from the group consisting of:

chr6:154039662 of gene OPRM1 118A>G;

chr19:41006936 of gene CYP2B6*13*6*7*9+516G>T;

chr22:42130692 of gene CYP2D6*4*10*1 4A+100C>T;

chr1:11796321 of gene MTHFR 677C>T;

CYP2C9 non EM (IM or PM); and/or

chr7:99768693 of gene CYP3A4*22 intron6 15389C>T.

In some embodiments, the opioid addiction risk is opioid use disorder (OUD) or relapse risk.

In further embodiments, the subject is a female or male.

### BRIEF DESCRIPTION OF FIGURES

The following figures are provided by way of example and are not intended to limit the scope of the invention.

FIG. 1 plots an opioid use disorder receiver operating characteristic curve for a female using a sex-stratified single count SNP Model 1.

FIG. 2 plots an opioid use disorder receiver operating characteristic curve for a male using a sex-stratified single count SNP Model 1.

FIG. 3 plots a relapse receiver operating characteristic curve for a female using a sex-stratified single count SNP Model 1.

FIG. 4 plots a relapse receiver operating characteristic curve for a male using a sex-stratified single count SNP Model 1.

### DETAILED DESCRIPTION

### Definitions

Unless defined otherwise, all technical and scientific terms used herein have the same meaning as commonly understood by one of ordinary skill in the art to which this disclosure belongs. Accordingly, the following terms are intended to have the following meanings:

As used in the specification and claims, the singular form “a”, “an” and “the” includes plural references unless the context clearly dictates otherwise.

As used herein, “administration” of a disclosed compound encompasses the delivery to a subject of a compound as described herein, or a prodrug or other pharmaceutically acceptable derivative thereof, using any suitable formulation or route of administration, e.g., as described herein.

As used herein, the term “and/or,” when used in a list of two or more items, means that any one of the listed items can be employed by itself, or any combination of two or more of the listed items can be employed. For example, if a composition is described as comprising components A, B, and/or C, the composition can contain A alone; B alone; C alone; A and B in combination; A and C in combination; B and C in combination; or A, B, and C in combination.

As used herein, “treatment” and “treating”, are used interchangeably herein, and refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. The term “treat”, in all its verb forms, is used herein to mean to relieve, alleviate, prevent, and/or manage at least one symptom of a disorder in a subject.

As used herein, “subject” or “patient” to which administration is contemplated includes, but is not limited to, humans (i.e., a male or female of any age group, e.g., a pediatric subject (e.g., infant, child, adolescent) or adult subject.

As used herein, “opioid use disorder” is a problematic pattern of opioid use that causes significant impairment or distress. A diagnosis is based on specific criteria such as unsuccessful efforts to cut down or control use, or use resulting in social problems and a failure to fulfill obligations at work, school, or home, among other criteria. Opioid use disorder has also been referred to as “opioid abuse or dependence” or “opioid addiction.”

As used herein, “relapse risk” is the risk of recurrence of opioid use disorder that has gone into remission or recovery. During the recovery process, subjects may become exposed to certain triggers or have genomic predisposition that increase the risk of returning to opioid use disorder or addiction.

Deoxyribonucleic acid “DNA” is a molecule composed of two chains that coil around each other to form a double helix carrying the genetic instructions used in the growth, development, functioning, and reproduction of all known organisms. DNA and ribonucleic acid (RNA) are nucleic acids; alongside proteins, lipids and complex carbohydrates (polysaccharides), nucleic acids are one of the four major types of macromolecules that are essential for a subject's functioning and development.

The two DNA strands are also known as polynucleotides as they are composed of simpler monomeric units called nucleotides. Each nucleotide is composed of one of four nitrogen-containing nucleobases (cytosine [C], guanine [G], adenine [A] or thymine [T]), a sugar called deoxyribose, and a phosphate group. The nucleotides are joined to one another in a chain by covalent bonds between the sugar of one nucleotide and the phosphate of the next, resulting in an alternating sugar-phosphate backbone. The nitrogenous bases of the two separate polynucleotide strands are bound together, according to base pairing rules (A with T and C with G), with hydrogen bonds to make double-stranded DNA.

A single-nucleotide polymorphism “SNP” is a substitution of a single nucleotide that occurs at a specific position in the genome, where each variation is present to some appreciable degree within a population. For example, at a specific base position in the human genome, the C nucleotide may appear in most individuals, but in a minority of individuals, the position is occupied by an A. This means that there is a SNP at this specific position, and the two possible nucleotide variations—C or A—are said to be alleles for this position. For purposes of this disclosure, in certain embodiments, “allele” refers to genetic material, including, but not limited to, one or more DNA fragments, present in biological samples, in vitro, corresponding to one or both alleles of a SNP at a specific position. SNPs denote differences in a subject's susceptibility or risk to a wide range of diseases including opioid use disorders and relapse risk. The severity of risks and the way the body responds to treatments are also manifestations of genetic variations.

### Pharmacogenomic Testing for Opioid Addiction

Genetics plays an important role in how an individual metabolizes and responds to medications, including opioids prescribed for pain management and those used for medication assisted treatment (MAT) of opioid use disorder (OUD). With a high rate of opioid and OUD medication use, solutions for improving prescribing, treatment, and prevention are in great need.

Precision Medicine is an approach to patient care that describes a paradigm in which treatment and prevention plans are tailored to incorporate the individual's genetic variability. Pharmacogenomics (PGX) is at the forefront of precision medicine. PGX applies the knowledge of an individual's genetics to drug response and helps determine if the patient will have an adverse or therapeutic response to a particular medication. It is estimated that 20 to 95% of the variability in a patient's response to drugs is associated with genetics. If a patient has a genetic variant, the drug may be metabolized too slowly (causing toxic levels to build up) or too quickly (resulting in a lack of therapeutic efficacy). PGX testing provides the genetic information necessary to direct more accurate prescribing for each patient.

Pharmacogenomic testing provides valuable information regarding an individual's ability to respond to specific drugs. Despite the potential to improve healthcare quality and reduce costs, implementation into routine clinical practice has been slow. This is in large part, due to the lack of studies that assess clinical utility. Early evidence suggests that genetic variability plays a role in the response to addiction treatment medications. For example: 1) genetic mutations in OPRM1 are associated with the efficacy of naltrexone (VIVITROL®), 2) genetic variability in the CYP2B6 enzyme is associated with methadone plasma concentrations and clearance, and 3) buprenorphine (SUBOXONE®) efficacy is associated with mutations in OPRD1. In addition, the efficacy of buprenorphine (SUBOXONE®) may be further reduced if the patient is taking other medications that work through the same metabolic pathways or have a genetic aberration in specific metabolizing enzymes. PGX analysis may help identify the most effective anti-addictive medication for each patient and improve the long-term success of recovery.

As disclosed herein, the examples demonstrates the relationship between mutations in specific drug metabolizing genes and addiction recovery. Given the limited treatment options and low treatment success rates, improved methods for treating a growing population health problem such as OUD are in great need.

In addition to the genes that regulate opioid metabolism and drug efficacy, other genes related to addiction risk have been identified. It is believed that up to 50% of addiction is related to genetics. Understanding a patient's genetic predisposition or susceptibility to addiction may be useful for: 1) helping addicts understand their disease has a genetic component; 2) shifting blame and stigma to a genetic predisposition may help to improve addiction treatment success; and 3) identifying patients at risk of developing an addiction and preventing the growth of OUD and relapse.

The disclosure herein demonstrates PGX testing can improve initial opioid prescribing practices for MAT of OUD and the relationship between mutations in specific drug metabolizing genes and addiction recovery. This approach includes analysis of addiction risk genes in all patients recruited to validate their association in a clinical population. These genes may be useful for identifying patients at risk for addiction at the initial point of prescribing and for identifying OUD patients who may face greater recovery challenges because of their susceptibility to relapse. The addiction risk panel provided in Table 1 contains 180 addiction risk mutations, including single nucleotide polymorphisms (SNPs). SNPs are the most common type of genetic variation among people and represent a difference in a single DNA nucleotide. For example, a SNP may replace the nucleotide cytosine (C) with the nucleotide thymine (T) in a given stretch of DNA.

The scoring SNP Models and algorithms disclosed herein could be used as tools when a health care team is making a treatment plan for a patient who will be prescribed opioids (addiction risk) or will be treating an addiction (relapse risk). Possible benefits to knowing the following levels of risk may include:

High risk of OUD: Evaluate the risk and benefits to prescribing opioids, increase caution about the quantities of opioids prescribed and dispensed; increase monitoring by a health care professional between visits, assess for addiction more frequently; include a conservative time frame for opioid use; intentionally tapering off the opioid and providing resources for patients with high risk of OUD; consider the use of non-opioid therapies (i.e. adjuvant therapies), alone or in combination, depending on the type & source of pain.

Low risk of OUD: As low risk does not mean no risk, caution should be given to interpreting low risk as this does not mean that opioids can be freely used or that caution should be reduced from current levels. Evaluate the risk and benefits to prescribing opioids; establish a monitoring plan, which may be less frequent than someone at high risk of OUD; minimize monitoring of addiction over time to save on health care resources; increase caution about the amount of opioids prescribed initially and between visits. While someone may have a low risk of OUD, it is known that prescribing opioids can lead to increased tolerance, dependence and addiction; therefore, the use of non-opioid therapies (i.e. adjuvant therapies), alone or in combination, depending on the type & source of pain, can be considered.

High risk of relapse: Potentially justify a longer inpatient rehabilitation stay, a longer duration of intensive outpatient rehabilitation; potentially help guide a patient to know that extra work must be done.

Low risk of relapse: As low risk does not mean no risk, caution should be given to interpreting low risk (MAT may still require monitoring/support), or that caution should be reduced from current levels. Potentially justify an earlier transition from inpatient rehabilitation to intensive outpatient rehabilitation.

TABLE 1
Full Opioid Panel having 180 SNPs

| | Assay Names used | NCBI SNP | Gene | | |
|---|---|---|---|---|---|
| | in Addiction Panel | Reference | Symbol | CHR | Context Sequence [VIC/FAM] |
| 1 | ABCB1/ MDR1, | rs1045642 | ABCB1/ | 7 | TGTTGGCCTCCTTTGCTGCCCTCAC[A/G]A |
| | c.3435T > C | | MDR1 | | TCTCTTCCTGTGACACCACCCGGC |
| 2 | ANKK1, c.1469A > G | rs2734849 | ANKK1 | 11 | TCCCGTCAGGCTGACCCCAACCTGC[A/G]T |
| | | | | | GAGGCTGAGGGCAAGACCCCCCTC |
| 3 | ANKK1/DRD2, | rs1800497 | ANKK1/ | 11 | CACAGCCATCCTCAAAGTGCTGGTC[A/G] |
| | 17316G > A Taq1A | | DRD2 | | AGGCAGGCGCCCAGCTGGACGTCCA |
| 4 | ARHGAP28, | rs2567261 | ARHGAP | 18 | GAACCACTGGCAGGTACACTTTAAA[C/T] |
| | c.334 + 692T > C | | 28 | | GGGTGAATCGAATGGCATGTGAAGT |
| 5 | AUTS2, c.661- | rs6943555 | AUTS2 | 7 | AGCCCTCATTCTAATAGTAAGGCTG[A/T]T |
| | 94715T > A | | | | TTCCTCTTTTCCAATGTTTATGTA |
| 6 | BDNF, c.196G > A | rs6265 | BDNF | 11 | TCCTCATCCAACAGCTCTTCTATCA[C/T]G |
| | | | | | TGTTCGAAAGTGTCAGCCAATGAT |
| 7 | BDNF, c.−21- | rs11030104 | BDNF | 11 | ATTAAAAAGCAGATAACACTACCAC[A/G] |
| | 4385T > C | | | | TACTAACTGTCCTACAATTTCCTGT |
| 8 | BDNF, c.− | rs10767664 | BDNF | 11 | GTAGGCTTGACATTGACATGTTTTT[A/T]C |
| | 22 + 16205A > T | | | | TATTAATAATTTTAATTGGCTGAG |
| 9 | BDNF, c.25- | rs16917234 | BDNF | 11 | CTCTTGAACTCAGTCCTGAAAATAA[C/T]G |
| | 18242A > G | | | | TTAATAGCTGAGAAAAGAGCATTG |
| 10 | BDNFOS/antiBDNF, | rs988712 | BDNFOS/ | 11 | ATTCTGGAATTTATATGAAAAGACC[G/T]T |
| | n.144 + 1719G > T | | antiBDNF | | ATAGCATAGGGACAATAGTTAAAA |
| 11 | BDNFOS/antiBDNF, | rs7481311 | BDNFOS/ | 11 | TCCATTGGTCATGTCAGCACTGCTA[T/C]T |
| | n.144 + 21466T > C | | antiBDNF | | GTTGGGCTCAAAGGCTGAGATAGT |
| 12 | BDNFOS/antiBDNF, | rs11030096 | BDNFOS/ | 11 | TACACAGGTGAATGAAAATGTCCAC[C/T] |
| | n.305 + 3991T > C | | antiBDNF | | GCTCTAGAAGAGTTTATACAAATAA |
| 13 | CACNA2D2, | rs5030977 | CACNA2 | 3 | TGTACTGGGCCCAGGTCAGGGTAGC[C/T] |
| | c.1260 + 22G > A | | D2 | | CCTGCCTCGGTTGAGCCTCACCGTC |
| 14 | CNIH3, | rs1369846 | CNIH3 | 1 | CAGGCAATGACGCACATAGCATCCT[C/T] |
| | c.198 + 21550C > T | | | | GCCTGTTCCGGAGGGTCGCCTTTGA |
| 15 | CNIH3, | rs1436171 | CNIH3 | 1 | AGAGCTTCCACCCAGAGAAGTTGAC[A/G] |
| | c.198 + 9283A > G | | | | GCAGACAGATGTTGCCAGCTGCCAG |
| 16 | CNIH3, c.199- | rs1436175 | CNIH3 | 1 | TCATCTGCCCCGTGCTGAGTAACTA[A/G]A |
| | 9798G > A | | | | GGCAGAAGATGACCTGTTTCTGCC |
| 17 | CNIH3, | rs10799590 | CNIH3 | 1 | CCCCCTGCCCAGTTACCCTGCATCT[A/G]T |
| | c.81 + 17525G > A | | | | TCTGTGTTGAGCAGAGGTGTTCAA |
| 18 | CNIH3, | rs12130499 | CNIH3 | 1 | GTCTGCAGTCAGATTAGTAACTATT[C/T]C |
| | c.81 + 31557C > T | | | | TTCCTGGTAGGACAAGAGCAAAAG |
| 19 | CNIH3, c.82- | rs298733 | CNIH3 | 1 | AAACTAGGTGTGACCTCAGTAGACA[A/C] |
| | 26409C > A | | | | TGATTTTAGCCACTGTTGATGCCTG |
| 20 | CNR1, c.−63- | rs2023239 | CNR1 | 6 | TAGGTTTGTGGATGTGCCAGGACCA[C/T] |
| | 5426A > G | | | | GTAAGGAACAGCTCTCTCATATATT |
| 21 | CNR1, c.−63- | rs806379 | CNR1 | 6 | GCAGAACTGATCTGAAATTAGATGA[A/T] |
| | 6211T > A | | | | ATTAAATGCATGTAAAACATAGTGC |
| 22 | CNR1, c.−63- | rs6928499 | CNR1 | 6 | TGAAATTAAATGCATGTAAAACATA[C/G] |
| | 6233C > G | | | | TGCCTGACACAAAAGTAAGTCTTCA |
| 23 | COMT, 472G > A | rs4680 | COMT | 22 | CCAGCGGATGGTGGATTTCGCTGGC[A/G] |
| | | | | | TGAAGGACAAGGTGTGCATGCCTGA |
| 24 | CREBBP, c.3837- | rs3025684 | CREBBP | 16 | TCCTTGCAATCAACGAAACTAGGAG[A/G] |
| | 8C > T | | | | CAAAGAAGGCGCACTGTTAAAGCAC |
| 25 | CSNK1E, c.737- | rs1534891 | CSNK1E | 22 | AGAGCCATGGCCTTCCCTATCCTAC[C/T]G |
| | 160A > G | | | | TGATGAAAGCCTAGCCTGCCCGTG |
| 26 | CSNK1E, c.77- | rs6001093 | CSNK1E | 22 | ATTGCCTTATAGCCTTGGGGTTAGG[C/T]A |
| | 2140G > A | | | | AAGCTTTTATTTTTATCCCTGATT |
| 27 | CSNK1E, | rs135745 | CSNK1E | 22 | ACTAGGCCTCTCACACTGGATTCTG[C/G]A |
| | g.38287631G > C | | | | TTGGGGTGAACCACTTGCTACTCT |
| 28 | CYP1A2, *1C; L - | rs2069514 | CYP1A2 | 15 | TGGCTCACCGCAACCTCCGCCTCTC[G/A]G |
| | 3860G > A | | | | ATTCAAGCAATTGTCATGCCCCAG |
| 29 | CYP1A2, *1D; L; V; | rs35694136 | CYP1A2 | 15 | TGCAGTGAGCCATGATTGTGGCACA[T/-]G |
| | W −2467; −1635delT | | | | AACCCCAACCTGGGTGACAGAGCA |
| 30 | CYP1A2, *1F, K, | rs762551 | CYP1A2 | 15 | TGCTCAAAGGGTGAGCTCTGTGGGC[C/A] |
| | L; V; W + −163A > C | | | | CAGGACGCATGGTAGATGGAGCTTA |
| 31 | CYP1A2, *1K - | rs12720461 | CYP1A2 | 15 | GGCTAGGTGTAGGGGTCCTGAGTTC[C/T] |
| | 729C > T | | | | GGGCTTTGCTACCCAGCTCTTGACT |
| 32 | CYP2B6, | rs3745274 | CYP2B6 | 19 | TCATGGACCCCACCTTCCTCTTCCA[G/T]T |
| | *13*6*7*9 + 516G > T | | | | CCATTACCGCCAACATCATCTGCT |
| 33 | CYP2C19, *17*4B - | rs12248560 | CYP2C19 | 10 | AAATTTGTGTCTTCTGTTCTCAAAG[C/T]A |
| | 806C > T | | | | TCTCTGATGTAAGAGATAATGCGC |
| 34 | CYP2C19, *2 + | rs4244285 | CYP2C19 | 10 | TTCCCACTATCATTGATTATTTCCC[A/G]G |
| | 681G > A | | | | GAACCCATAACAAATTACTTAAAA |
| 35 | CYP2C19, *3 | rs4986893 | CYP2C19 | 10 | ACATCAGGATTGTAAGCACCCCCTG[A/G] |
| | 636G > A | | | | ATCCAGGTAAGGCCAAGTTTTTTGC |
| 36 | CYP2C19, *4 1A > G | rs28399504 | CYP2C19 | 10 | GTCTTAACAAGAGGAGAAGGCTTCA[A/G] |
| | | | | | TGGATCCTTTTGTGGTCCTTGTGCT |
| 37 | CYP2C19, *5 | rs56337013 | CYP2C19 | 10 | CCTATGTTTGTTATTTTCAGGAAAA[C/T]G |
| | 1297C > T | | | | GATTTGTGTGGGAGAGGGCCTGGC |
| 38 | CYP2C19, *6 | rs72552267 | CYP2C19 | 10 | CGGCGTTTCTCCCTCATGACGCTGC[A/G]G |
| | 395G > A | | | | AATTTTGGGATGGGGAAGAGGAGC |
| 39 | CYP2C19, *7 | rs72558186 | CYP2C19 | 10 | TGCTTCCTGATCAAAATGGAGAAGG[A/T] |
| | 19294T > A | | | | AAAATGTTAACAAAAGCTTAGTTAT |
| 40 | CYP2C19, *8 | rs41291556 | CYP2C19 | 10 | AATCGTTTTCAGCAATGGAAAGAGA[C/T] |
| | 358T > C | | | | GGAAGGAGATCCGGCGTTTCTCCCT |
| 41 | CYP2C19, *9 | rs17884712 | CYP2C19 | 10 | ATGGGGAAGAGGAGCATTGAGGACC[A/G] |
| | 431G > A | | | | TGTTCAAGAGGAAGCCCGCTGCCTT |
| 42 | CYP2C9, *11 | rs28371685 | CYP2C9 | 10 | GATTGAACGTGTGATTGGCAGAAAC[T/C] |
| | 1003C > T | | | | GGAGCCCCTGCATGCAAGACAGGAG |
| 43 | CYP2C9, *2 + | rs1799853 | CYP2C9 | 10 | GATGGGGAAGAGGAGCATTGAGGAC[C/T] |
| | 430C > T | | | | GTGTTCAAGAGGAAGCCCGCTGCCT |
| 44 | CYP2C9, *3 + | rs1057910 | CYP2C9 | 10 | TGTGGTGCACGAGGTCCAGAGATAC[C/A] |
| | noamp*4*4 1075A > C | | | | TTGACCTTCTCCCCACCAGCCTGCC |
| 45 | CYP2C9, *4 NOAMP | rs56165452 | CYP2C9 | 10 | GTGGTGCACGAGGTCCAGAGATACA[C/T] |
| | *3*3 1076T > C | | | | TGACCTTCTCCCCACCAGCCTGCCC |
| 46 | CYP2C9, *5 | rs28371686 | CYP2C9 | 10 | TGCACGAGGTCCAGAGATACATTGA[C/G] |
| | 1080C > G | | | | CTTCTCCCCACCAGCCTGCCCCATG |
| 47 | CYP2C9, *6 818delA | rs9332131 | CYP2C9 | 10 | TGATTGCTTCCTGATGAAAATGGAG[-/A] |
| | | | | | AGGTAAAATGTAAACAAAAGCTTAG |
| 48 | CYP2D6, *12 | rs5030862 | CYP2D6 | 22 | TCCACATGCAGCAGGTTGCCCAGCC[C/T] |
| | 124G > A | | | | GGGCAGTGGCAGGGGGCCTGGTGGG |
| 49 | CYP2D6, *14 NOAMP | rs5030865 | CYP2D6 | 22 | TTGTGCCCTTCTGCCCATCACCCAC[T/C]G |
| | *8*8 1758G > A | | | | GAGTGGTTGGCGAAGGCGGCACAA |
| 50 | CYP2D6, *17*40 | rs28371706 | CYP2D6 | 22 | ACGCGGCCCGAAACCCAGGATCTGG[G/A] |
| | 1023C > T | | | | TGATGGGCACAGGCGGGCGGTCGGC |
| 51 | CYP2D6,*2*4k*8*11 | rs16947 | CYP2D6 | 22 | GAGAACAGGTCAGCCACCACTATGC[A/G] |
| | *14*17*29*41 + 2850 | | | | CAGGTTCTCATCATTGAAGCTGCTC |
| | C > T | | | | |
| 52 | CYP2D6, *29 + | rs59421388 | CYP2D6 | 22 | TCTGGTCGCCGCACCTGCCCTATCA[C/T]G |
| | 3183G > A | | | | TCGTCGATCTCCTGTTGGACACGG |
| 53 | CYP2D6, *2A*35 + - | rs1080985 | CYP2D6 | 22 | TAATTTTGTATTTTTTGTAGAGACC[G/C]G |
| | 1584C > G | | | | GTTCTTCCAAGTTGTCCAGGCTGG |
| 54 | CYP2D6, *3 | rs35742686 | CYP2D6 | 22 | GGCTGGGCTGGGTCCCAGGTCATCC[T/-]G |
| | 2549delA | | | | TGCTCAGTTAGCAGCTCATCCAGC |
| 55 | CYP2D6, *35 31G > A | rs769258 | CYP2D6 | 22 | AGGAGCAGGAAGATGGCCACTATCA[C/T] |
| | | | | | GGCCAGGGGCACCAGTGCTTCTAGC |
| 56 | CYP2D6, *4 | rs3892097 | CYP2D6 | 22 | AGACCGTTGGGGCGAAAGGGGCGTC[C/T] |
| | 1846G > A | | | | TGGGGGTGGGAGATGCGGGTAAGGG |
| 57 | CYP2D6, *4*10*14A + | rs1065852 | CYP2D6 | 22 | CCGGGCAGTGGCAGGGGGCCTGGTG[A/G] |
| | 100C > T not*4M | | | | GTAGCGTGCAGCCCAGCGTTGGCGC |
| 58 | CYP2D6, *41 + | rs28371725 | CYP2D6 | 22 | TTCATGGGCCCCCGCCTGTACCCTT[C/T]C |
| | 2988G > A | | | | TCCCTCGGCCCCTGCACTGTTTCC |
| 59 | CYP2D6, *6 | rs5030655 | CYP2D6 | 22 | AGGCAGGCGGCCTCCTCGGTCACCC[A/-]C |
| | 1707delT | | | | TGCTCCAGCGACTTCTTGCCCAGG |
| 60 | CYP2D6, *7 | rs5030867 | CYP2D6 | 22 | GATGGGCTCACGCTGCACATCCGGA[G/T] |
| | 2935A > C | | | | GTAGGATCATGAGCAGGAGGCCCCA |
| 61 | CYP2D6, *8NOAMP | rs5030865 | CYP2D6 | 22 | TTGTGCCCTTCTGCCCATCACCCAC[A/C]G |
| | *14*14 1758G > T | | | | GAGTGGTTGGCGAAGGCGGCACAA |
| 62 | CYP2D6, *9 | rs5030656 | CYP2D6 | 22 | CCCCACCGTGGCAGCCACTCTCAC[CTT/-] |
| | 2613_2615delAGA | | | | CTCCATCTCTGCCAGGAAGGCCTC |
| 63 | CYP3A4, *12 | rs12721629 | CYP3A4 | 7 | ACATCTTTTTTGCAGACCCTCTCAA[A/G]T |
| | 1117C > T | | | | CTCATAGCAATTGGGAATAATCTG |
| 64 | CYP3A4, *17 | rs4987161 | CYP3A4 | 7 | GTTGAGAGAGTCGATGTTCACTCCA[A/G] |
| | 566T > C | | | | ATGATGTGCTAGTGATCACATCCAT |
| 65 | CYP3A4, *1B + - | rs2740574 | CYP3A4 | 7 | TAAAATCTATTAAATCGCCTCTCTC[C/T]T |
| | 392A > G | | | | GCCCTTGTCTCTATGGCTGTCCTC |
| 66 | CYP3A4, *2 664T > C | rs55785340 | CYP3A4 | 7 | GAAATAGTAGTCCACATACTTATTG[A/G] |
| | | | | | GAGAAAGAATGGATCCAAAAAATCA |
| 67 | CYP3A4, *22 intron6 | | | | GTGCCAGTGATGCAGCTGGCCCTAC[G/A] |
| | 15389C > T | rs35599367 | CYP3A4 | 7 | CTGGGTGTGATGGAGACACTGAACT |
| 68 | CYP3A4, *3 | rs4986910 | CYP3A4 | 7 | TTTCATGTTCATGAGAGCAAACCTC[A/G]T |
| | 1334T > C | | | | GCCAATGCAGTTTCTGGGTCCACT |
| 69 | CYP3A5, *2 | rs28365083 | CYP3A5 | 7 | CTTTGGGTCATGGTGAAGAGCATAA[G/T] |
| | 27289C > A | | | | TTGGAATCACCACCATTGACCCTTT |
| 70 | CYP3A5, *3*10*1D | rs15524 | CYP3A5 | 7 | AGCTTTCTTGAAGACCAAAGTAGAA[A/G] |
| | 31611C > T | | | | TCCTTAGAATAACTCATTCTCCACT |
| 71 | CYP3A5, *3*9 | rs776746 | CYP3A5 | 7 | ATGTGGTCCAAACAGGGAAGAGATA[T/C] |
| | 6986A > G | | | | TGAAAGACAAAAGAGCTCTTTAAAG |
| 72 | CYP3A5, *3B H30Y | rs28383468 | CYP3A5 | 7 | ATTCCCAGTCTCTTAAAAAGTCCAT[G/A]T |
| | 3705C > T | | | | GTACGGGTCCCATATCTACAAAGT |
| 73 | CYP3A5, *6 | rs10264272 | CYP3A5 | 7 | CTAAGAAACCAAATTTTAGGAACTT[C/T]T |
| | 14690G > A | | | | TAGTGCTCTCCACAAAGGGGTCTT |
| 74 | CYP3A5, *7 | rs41303343 | CYP3A5 | 7 | CCATCTGTACCACGGCATCATAGGT[A/-]A |
| | 27131_27132insT | | | | GGTGGTGCCTGGAAGGAAAGAAAC |
| 75 | CYP3A5, *8 | rs55817950 | CYP3A5 | 7 | AGTCTCTTAAAAAGTCCATGTGTAC[A/G]G |
| | 3699C > T | | | | GTCCCATATCTACAAAGTGAAACA |
| 76 | CYP3A5, *9 | rs28383479 | CYP3A5 | 7 | CCCCTCACCTTATTGGGCAAAACTG[C/T]A |
| | 19386G > A | | | | TCAATCTCCTTTTGCAGTTTCTGC |
| 77 | DBH, c.−979T > C | rs1611115 | DBH | 9 | AAGGCAGCTGCCCTCAGTCTACTTG[C/T]G |
| | | | | | GGAGAGGACAGGAGGGAGAGGTGC |
| 78 | DBI, c.− | rs12613135 | DBI | 2 | CATAAACAGAGCTGAGGATCTTGCA[C/T] |
| | 216 + 1593A > G | | | | TCTCAGAATTATGAAAAGCAATATT |
| 79 | DCC, c.*2140T > C | rs2292043 | DCC | 18 | AGATTTTAGGGATTGAGTCACACCT[T/C]C |
| | | | | | AATCTATAGAATGAAGTTGACCAA |
| 80 | DCC, c.*3949T > C | rs12607853 | DCC | 18 | TACAGAAAAGCTTTTTATTTGAGTC[C/T]A |
| | | | | | GTGTTTAAAATTAAATTGGATACT |
| 81 | DCC, c.*4376T > C | rs16956878 | DCC | 18 | GGGCATGGGCCAAGGGATCTCACTG[C/T] |
| | | | | | GTGCTGAACATGTATTTTCAGATGC |
| 82 | DRD2, | rs2075652 | DRD2 | 11 | GCACTTAGTAAGCACTTTACAAATG[G/A]T |
| | c.285 + 191C > T | | | | AGTTGGGATTATTAAGGAAACAAT |
| 83 | DRD2, | rs2734833 | DRD2 | 11 | TCCTTCCTCTTTATCCCAAGGGGGC[G/A]G |
| | c.285 + 2169C > T | | | | TGAATAGGAAAGACAGAGTCCTCC |
| 84 | DRD2, c.−31- | rs4436578 | DRD2 | 11 | TCACACTGCTGGAAACCTCCGGAAG[C/T] |
| | 11361G > A | | | | CCTTGTCCCCACGTTTCTCATCCTT |
| 85 | DRD2, c.−31- | rs1079596 | DRD2 | 11 | CCAAAAATGTAGGGTATGGCAGTAA[C/T] |
| | 1215G > A | | | | GTTGAGGATAATTAAACTGCAGGGA |
| 86 | DRD2, c.−31- | rs17115583 | DRD2 | 11 | GGAATTGAAGAAGGTGTGTCAATGC[A/G] |
| | 13498C > T | | | | TCCTATTTTTATTGTTTTTTTTTTA |
| 87 | DRD2, c.−31- | rs11214607 | DRD2 | 11 | GGTAGCCTATGGACCACATTTAGCT[G/T]G |
| | 16735A > C | | | | CATACAGGATTTGTTGGGCTCACA |
| 88 | DRD2, c.−31- | rs1125394 | DRD2 | 11 | AATTAAACTTATCAGCATTCCAAGG[C/T]G |
| | 1781A > G | | | | TTTCATACAAAGCACATGACTTCC |
| 89 | DRD2, c.−31-882G > A | rs1079597 | DRD2 | 11 | GAACCACATGATCAGATTCGCCTTT[C/T]G |
| | Taq1B | | | | AATAGGTGATTCTGACAGCACTGT |
| 90 | DRD2, c.−585A > G | rs1799978 | DRD2 | 11 | GCGCTCCCACCCACACCCAGAGTAA[C/T] |
| | | | | | AAGCTGTGATTGCAGGCTGGGTCCT |
| 91 | DRD2, c.724- | rs2283265 | DRD2 | 11 | AGGAAACAGGCTCATAGAAGGTAAG[A/C] |
| | 353G > T | | | | AACTTGCCTAAGGTCACTCAGCAAA |
| 92 | DRD2, c.811-83G > T | rs1076560 | DRD2 | 11 | CCCATCTCACTGGCCCCTCCCTTTC[A/C]C |
| | | | | | CCTCTGAAGACTCCTGCAAACACC |
| 93 | DRD2, c.939T > C | rs6275 | DRD2 | 11 | GGCTGTCGGGAGTGCTGTGGAGACC[A/G] |
| | | | | | TGGTGGGACGGGTCGGGGAGAGTCA |
| 94 | DRD2, c.957C > T | rs6277 | DRD2 | 11 | TCTTCTCTGGTTTGGCGGGGCTGT[A/G]G |
| | | | | | GAGTGCTGTGGAGACCATGGTGGG |
| 95 | DRD3, c.−155- | rs9825563 | DRD3 | 3 | AATAGAAGAGAAGCAGGGTAAATGA[A/G] |
| | 2597T > C | | | | GTGATCCTTTCTCTCTGGACTTCAC |
| 96 | DRD3, c.25G > A | rs6280 | DRD3 | 3 | GCCCCACAGGTGTAGTTCAGGTGGC[C/T] |
| | | | | | ACTCAGCTGGCTCAGAGATGCCATA |
| 97 | DRD3, c.271- | rs324029 | DRD3 | 3 | ATAGGGAAGTGTTAGGTGAGGAGGG[A/G] |
| | 2909T > C | | | | TAGTTGTTGGAAAAGGGATGGAAGT |
| 98 | DRD3, c.527-630T > C | rs9288993 | DRD3 | 3 | AAAAGGCAGGTAATGATATTGTGAC[A/G] |
| | | | | | TGGAGAATGTGCACTTAGAAGGGTC |
| 99 | DRD3, | rs2654754 | DRD3 | 3 | CTCTGTCCATGTGTGTTCCCTTGAC[A/G]T |
| | c.723 + 2551C > T | | | | CTGTTTCCTCTAATGCAGGTGGCC |
| 100 | DRD4, c.−521T > C | rs1800955 | DRD4 | 11 | GGGCAGGGGGAGCGGGCGTGGAGGG[C/T] |
| | | | | | GCGCACGAGGTCGAGGCGAGTCCGC |
| 101 | EXOC4, c.1183- | rs718656 | EXOC4 | 7 | GGAGATAGAATTTGCTCTTGCTATT[C/T]A |
| | 39230C > T | | | | TTTCAAGAAATTGGTGATCTTGCA |
| 102 | FAAH, | rs2295633 | FAAH | 1 | GATGTTGTCGTCGGGGTGAACTGTG[A/G] |
| | c.1077 + 127A > G | | | | CCCTGTGGGACAAGTATATAGAGGG |
| 103 | FAAH, c.196- | rs3766246 | FAAH | 1 | AAAGAATATATCAAGAGGATTATCT[A/G] |
| | 2092A > G | | | | GTGTGTTTGGGGAGAAGTCTTGAAC |
| 104 | FAAH, c.385C > A | rs324420 | FAAH | 1 | CTGTGAGACTCAGCTGTCTCAGGC[A/C]C |
| | | | | | AAGGCAGGGCCTGCTCTATGGCGT |
| 105 | FSTL4, c.161- | rs31347 | FSTL4 | 5 | CATCCAAATGCTGACATGGCAAGGA[C/T] |
| | 71797G > A | | | | ATCTTGGTCATCAAGTCTAACCCCA |
| 106 | GABRB3, c.− | rs4906902 | GABRB3 | 15 | TCACGTTGGCATGTTTCTGTGCATT[A/G]A |
| | 1659T > C | | | | TTTTAAATATACTGCCTTTTTAAA |
| 107 | GABRB3, c.249- | rs7165224 | GABRB3 | 15 | TCTTCTTATTTTTCTTCTGTTCTCC[T/C]TC |
| | 6417A > G | | | | CCCTCCCCTCTCTTCCTTTTCTT |
| 108 | GAD1, | rs2058725 | GAD1 | 2 | CAGAGAGATGAGAACTACATCATTT[C/T] |
| | c.547 + 2419T > C | | | | ATTATGAAAGCCCAGAATGGCGTTG |
| 109 | GAD1, c.− | rs1978340 | GAD1 | 2 | CACCTTGACTGACCACGTTTTAGGC[A/G]T |
| | 64 + 189G > A | | | | GAAGATCTCCCCGCAGCCCGTTTG |
| 110 | GAL, c.−1998C > T | rs948854 | GAL | 11 | CACAGGAACGTGCCCTCTGCTCCTC[C/T]G |
| | | | | | CCTCTCGGCTGTCCTTCTGCCCAC |
| 111 | GAL, c.82-77G > A | rs694066 | GAL | 11 | ATTGTTCTAAGTCCTCTGCCATGCC[A/G]G |
| | | | | | GAAAGCCTGGGTGCACCCATTCAG |
| 112 | GR1K1, | rs2832407 | GR1K1 | 21 | ATCAAGATCAGAAAGTTACAACCCT[A/C] |
| | c.1251 + 1338G > T | | | | GGGAGTGTGTGTGGCTCTTGCAGTT |
| 113 | GRIN3A, | rs17189632 | GRIN3A | 9 | GTTTCCTGCTGCGCACTTCCCCTGA[A/T]A |
| | c.2766 + 7656A > T | | | | ATAAAATCACTGGAGAGTTTAATG |
| 114 | GRIN3B, c.1730C > T | rs2240158 | GRIN3B | 19 | TTTATGTGGCCCCTGCACTGGTCCA[C/T]G |
| | | | | | TGGCTGGGCGTCTTTGCGGCCCTG |
| 115 | HRPT2, c.1155- | rs1408830 | HRPT2 | 1 | GCACAAAATAGTGATGGCAATTCCT[A/G] |
| | 4716A > G | | | | GTTTCATCAGTTCTGTGAGATATGT |
| 116 | HTR2A, c.−998G > A, | rs6311 | HTR2A | 13 | ATGTCCTCGGAGTGCTGTGAGTGTC[C/T]G |
| | C > T | | | | GCACTTCCATCCAAAGCCAACAGT |
| 117 | HTR2A, c.102C > T, | rs6313 | HTR2A | 13 | ATGCATCAGAAGTGTTAGCTTCTCC[A/G]G |
| | G > A | | | | AGTTAAAGTCATTACTGTAGAGCC |
| 118 | HTR2C, −759C > T | rs3813929 | HTR2C | X | CTGCTCTTGGCTCCTCCCCTCATCC[C/T]G |
| | | | | | CTTTTGGCCCAAGAGCGTGGTGCA |
| 119 | HTR3B, c.*797T > A | rs1185027 | HTR3B | 11 | GTCAGCACAGGTTATTATTCACTTG[A/T]T |
| | | | | | GTGATTCCCATGGTCAACCTGGTA |
| 120 | HTR3B, | rs11606194 | HTR3B | 11 | AATTTGTTTATTAAAGCATCCTTTT[T/C]CT |
| | c.213 + 804T > C | | | | CCTATGTCTGAAAGATGGGCTGT |
| 121 | HTR3B, c.−381T > C | rs3758987 | HTR3B | 11 | TTAGTGTCCTGAATGTCAGCAAGAG[C/T] |
| | | | | | ACTGCCTTAGGTAAAGGCTGTAAAG |
| 122 | intergenic, | rs1986513 | intergenic | 4 | CTGTTCATGATTATGCTTAGTTTTA[A/T]CT |
| | g.125146073A > T | | | | CCACAGAATTGTTGCTGTGTTTC |
| 123 | intergenic, | rs10494334 | intergenic | 1 | TTAGTAGACTTGAATTATAGATGCC[A/G]C |
| | g.163535374G > A | | | | AACTCTCATTCATGTGCATTTCTG |
| 124 | intergenic, | rs966162 | intergenic | 12 | CAGTCTCCTAAGACTTCACCCTAAC[C/G]T |
| | g.18873522C > G | | | | TTTATTCAAGCCATCAGCTACCAA |
| 125 | intergenic, | rs965972 | intergenic | 1 | TTGTTAGGATTCACATTTAAGTGAC[A/G]T |
| | g.193494720G > A | | | | AAAAACTGAGAAGAGTTAAGCGGC |
| 126 | intergenic, | rs952985 | intergenic | 7 | CATCAATTCAGCTGCAGTATCTTCA[G/T]T |
| | g.9757835G > T | | | | TCTTACAGTGGGGAAGCCAGAATC |
| 127 | KCNC1, c.*1934A > C | rs60349741 | KCNC1 | 11 | ATGTGTTTGTTCAGACATGCACACC[A/C]G |
| | | | | | CTAATCCCAGGACACAAAACCTGT |
| 128 | LINC01456, | rs2213602 | LINC01456 | X | TCTAAGTGCTTTACAAACGTTGTTA[A/G]C |
| | g.18089955A > G | | | | TCATTTCACCCTTGCAACCATACG |
| 129 | IVIPDZ, c.184- | rs1389752 | IVIPDZ | 9 | TAGCTCCTGAACTAAATGAGACACA[A/T] |
| | 10705T > A | | | | AATGGAGCAATAAGTTATAAGAAGG |
| 130 | MTHFR, 1298A > C | rs1801131 | MTHFR | 1 | AAGAACGAAGACTTCAAAGACACTT[G/T] |
| | | | | | CTTCACTGGTCAGCTCCTCCCCCCA |
| 131 | MTHFR, 677C > T | rs1801133 | MTHFR | 1 | GAAAAGCTGCGTGATGATGAAATCG[G/A] |
| | | | | | CTCCCGCAGACACCTTCTCCTTCAA |
| 132 | NFKB1, c.119- | rs230530 | NFKB 1 | 4 | TTTTTAGCACCAAACATCTTAATTT[A/G]C |
| | 1025A > G | | | | ATTCAAATAAATGAGAACCACCAT |
| 133 | NR4A2, | rs1405735 | NR4A2 | 2 | ATAGCAGCCCGAATAAACTAAGAGA[C/G] |
| | g.156377320C > G | | | | GATACAATTTTAAAAAACAAATCCA |
| 134 | NRXN3, | rs11624704 | NRXN3 | 14 | TGTCCTCTGGGTATAATCTCACTTA[A/C]C |
| | c.757 + 21874A > C | | | | TTTACTCTGCAAATGCAATGTTGG |
| 135 | NTSR1, c.715- | rs3915568 | NTSR1 | 20 | TGCCTTGGATGCATCAGGTGCACCG[T/C] |
| | 16565T > C | | | | AGGGCTTTTGAAGGCTCCACGAGG |
| 136 | OPRD1, *343G > A | rs4654327 | OPRD1 | 1 | TTAAACAGGGCATCTCCAGGAAGGC[A/G] |
| | | | | | GGGCTTCAACCTTGAGACAGCTTCG |
| 137 | OPRD1, | rs6669447 | OPRD1 | 1 | CTCCTTCCCCCTTGCCTGGCAGATG[C/T]C |
| | c.227 + 10239T > C | | | | TGGACTTTGAGAGGCAGGGGGCTG |
| 138 | OPRD1, | rs2236857 | OPRD1 | 1 | GAGGGTCCAACACTCAGACAGCATG[C/T] |
| | c.227 + 22487T > C | | | | CACTAGGTGTTTGTACAAAGGACCT |
| 139 | OPRD1, | rs678849 | OPRD1 | 1 | GTCCTTCTTACCATAGTGTCAAAAG[C/T]A |
| | c.227 + 6066C > T | | | | CCTGCTAGGTGCTGAGCTTGGCTG |
| 140 | OPRD1, | rs2236861 | OPRD1 | 1 | GGGCGGCAGAGCATCCGGAGTGGCC[A/G] |
| | c.227 + 634G > A | | | | TCGTCCCTGTGTTTGTGCAGCTGTG |
| 141 | OPRD1, c.228- | rs10753331 | OPRD1 | 1 | GGAGTGATTAAATGAGGTGATCTCT[A/G] |
| | 20884G > A | | | | TAAATGTCATAGTAGCATTCGATAG |
| 142 | OPRD1, c.228- | rs508448 | OPRD1 | 1 | AAGGGTACAGCAGGGAACAAAATGG[A/G] |
| | 3941A > G | | | | 1CGAAGTCTTCTGGCTTCAGGGAACT |
| 143 | OPRD1, c.80G > T | rs1042114 | OPRD1 | 1 | GCCTCGGACGCCTACCCTAGCGCCT[G/T]C |
| | | | | | CCCAGCGCTGGCGCCAATGCGTCG |
| 144 | OPRD1, c.921C > T | rs2234918 | OPRD 1 | 1 | CGCTGCACCTGTGCATCGCGCTGGG[C/T]T |
| | | | | | ACGCCAATAGCAGCCTCAACCCCG |
| 145 | OPRK1, c.258- | rs6473797 | OPRK1 | 8 | AAAACACAAGTGTGATCAAATGCCA[C/T] |
| | 5311A > G | | | | GGACCCACAGGAAGCTGGTGGCTCT |
| 146 | OPRK1, c.36G > T | rs1051660 | OPRK1 | 8 | AGGCGCTCGGGGCGCAGGTAGGGCC[A/C] |
| | | | | | GGCTCCCCGCGGAAGATCTGGATCG |
| 147 | OPRM, | rs9479757 | OPRM | 6 | TGATGTTACCAGCCTGAGGGAAGGA[A/G] |
| | c.643 + 31G > A | | | | GGTTCACAGCCTGATATGTTGGTGA |
| 148 | OPRM1, c.1- | rs1074287 | OPRM1 | 6 | TGGTATTCTATTGTACTGTGGCTGA[A/G]G |
| | 11487A > G | | | | TAGTACTCAAACCACAAAATGCAG |
| 149 | OPRM1, | rs510769 | OPRM1 | 6 | TGGTGTTGATGTGTATATTCAAATA[C/T]T |
| | c.290 + 1050C > T | | | | ACATGTGAATGTGAAATGCCATAT |
| 150 | OPRM1, c .291- | rs1381376 | OPRM1 | 6 | GGAGAGGGATAAAAATGAGAATCAA[C/T] |
| | 17703 C > T | | | | GTGGGAATGGTAAGATAACAAGAGC |
| 151 | OPRM1, c.291- | rs563649 | OPRM1 | 6 | TTAGATCATGCAGGTCTATAACCAA[C/T]G |
| | 2994C > T | | | | GTGAATCTAGCAAAAGTTATTTTC |
| 152 | OPRM1, c.644- | rs2075572 | OPRM1 | 6 | GTTAGCTCTGGTCAAGGCTAAAAAT[C/G] |
| | 83 G > C | | | | AATGAGCAAAATGGCAGTATTAACA |
| 153 | OPRM1, | rs6848893 | OPRM1 | 4 | CTGGAAGAAAGTAACAGAAGATAAG[C/T] |
| | g.180916588C > T | | | | GGGGTGAGAGTGCTGGAGCAGTCAA |
| 154 | OPRM1, 118A > G | rs1799971 | OPRM1 | 6 | GGTCAACTTGTCCCACTTAGATGGC[A/G]A |
| | | | | | CCTGTCCGACCCATGCGGTCCGAA |
| 155 | PDYN, c.*1030C > T | rs2235749 | PDYN | 20 | GAGTCCCTTACCCAATGCCCAGTGC[A/G]T |
| | | | | | ATGTTGGGCCAGATGGCTTGGACT |
| 156 | PDYN, c.*743T > C | rs910080 | PDYN | 20 | TTTTCACTCCCTTCTGTAAGGAGTT[A/G]G |
| | | | | | GCACTGTCCAGGGTACCAACATGA |
| 157 | PDYN, c.−321A > G | rs1997794 | PDYN | 20 | CCCTTCAACTCGAACTCCCTGGGCC[C/T]G |
| | | | | | ACACAATAGGCTTCTCTTCGTGAC |
| 158 | PDYN, | rs1022563 | PDYN | 20 | CATCCACCACTACCACTGGCAGTGT[C/T]T |
| | g.1973693C > T | | | | GAGAGTCCTGATGCCTGTGGCTGT |
| 159 | PENK, c.−1973A > G | rs2609997 | PENK | 8 | TGTATCCAATCCACCTATGCATCTA[C/T]G |
| | | | | | TCTCCTAGACCTAGGGGGAAACCA |
| 160 | RGS9-2, c.−2050G > A | rs1530351 | RGS9-2 | 17 | TGGATGGATCTCTGCAGGGTCCAGC[A/G] |
| | | | | | TCCTCTAAATTGGAGGCTCTGAATC |
| 161 | SCN9A, c.3448C > T | rs6746030 | SCN9A | 2 | TTAACTTGGCAGCATGAGAACCTCC[A/G]T |
| | | | | | ACACAACCTGACAAGAAAGACATG |
| 162 | SLC6A3, c.−972T > C | rs2652511 | SLC6A3 | 5 | CAGCGCGCGGAGGAATGGAGCCCCC[A/G] |
| | | | | | GGCCGCCAAGGCCCAGGATGTCCAG |
| 163 | SLCO1B1, *1B | rs2306283 | SLCO1B1 | 12 | CAGGTATTCTAAAGAAACTAATATC[A/G] |
| | 388A > G | | | | ATTCATCAGAAAATTCAACATCGAC |
| 164 | SLCO1B1, *5 | rs4149056 | SLCO1B 1 | 12 | TCTGGGTCATACATGTGGATATATG[C/T]G |
| | 521T > C | | | | TTCATGGGTAATATGCTTCGTGGA |
| 165 | SORCS3, c.628- | rs728453 | SORCS3 | 10 | ACTGTCTTTGTCATTCCTGATCAGC[A/G]T |
| | 28349A > G | | | | GCCTCTGTGCCTTCATAGACGTTG |
| 166 | TACR1, c.333T > C | rs6715729 | TACR1 | 2 | TACTGGCGAAGACAGCGGCGATGGG[A/G] |
| | | | | | AAGAAGTTGTGGAACTTGCAGTAGA |
| 167 | TACR1, c.390- | rs735668 | TACR1 | 2 | ACCTCCCCTATATTCTCCCCTCTCC[A/C]TT |
| | 15150T > G | | | | TCGCATTCTGTTTCACCATCGTT |
| 168 | TACR1, | rs6741029 | TACR1 | 2 | AACTCCAAAACACACACTCTTCTAA[G/T]T |
| | c.584 + 2505A > C | | | | ATTATACTGCCTCAAAACAAGTTT |
| 169 | TACR3, | rs1384401 | TACR3 | 4 | GATAACCCATAGAGAACCTTTTTCA[A/G] |
| | c.888 + 12273C > T | | | | ATGATTGCCAAACACTGAAAGGCTT |
| 170 | TACR3, | rs4530637 | TACR3 | 4 | TAGTCAGTGTGGGTCCTGAGGTTGT[A/G]G |
| | g.103585232A > G | | | | CATGTTTAGCAAAGTTACAGAACA |
| 171 | TAOK3, | rs795484 | TAOK3 | 12 | AGACATGCGTGCCTTGGTGTTTCGG[C/T]C |
| | c.2535 + 170A > G | | | | TGTAGAAGGGGGACAATGCCTACC |
| 172 | UGT2B7, c.−161T > C | rs7668258 | UGT2B7 | 4 | CAGATCATTTACCTTCATTTGTCTC[C/T]TT |
| | | | | | GCCATCCACATGCTCAGACTGTT |
| 173 | UGT2B7, c.−327A > G | rs7662029 | UGT2B7 | 4 | TTGTGTCAAATGGACTGCAGAAACA[A/G] |
| | | | | | GATCTGTCACTGCTACTGTTCTGGA |
| 174 | UGT2B7, c.−900G > A | rs7438135 | UGT2B7 | 4 | CCAAATAACTGTGAGGAAGTGAGTC[A/G] |
| | | | | | GAGAACAAGCTAACCTAATGATTAA |
| 175 | VKORC1, *2 - | rs9923231 | VKORC1 | 16 | GATTATAGGCGTGAGCCACCGCACC[C/T] |
| | 1639G > A | | | | GGCCAATGGTTGTTTTTCAGGTCTT |
| 176 | WLS, c.107-301T > G | rs1036066 | WLS | 1 | CTTCAAAACAATGTCACAAAAAATC[A/C] |
| | | | | | ACTGTGCTACAGTTCCCACCTGATT |
| 177 | WLS, c.1393G > A | rs983034 | WLS | 1 | AAGGCACTGTTCACTTGGACTGTGA[C/T]G |
| | | | | | CCGCCCCATTTCCAATGGCCTTCC |
| 178 | WLS, c.432G > A | rs3748705 | WLS | 1 | GGGCCATTTCAGTCCACTCAGCAAA[C/T] |
| | | | | | GCGTCATCACGGTAAGCCAGGGAAA |
| 179 | ZNF804A, | rs7597593 | ZNF804A | 2 | ATTTATGAATTTAATTCATTAATGT[C/T]G |
| | c.111 + 69783T > C | | | | TAAATAGTATTGCCCGAGAATTGG |
| 180 | ZNF804A, c.256- | rs1344706 | ZNF804A | 2 | AGATATCCAAGAAGTTGATTCTGAT[A/C] |
| | 19902A > C | | | | GTTTTTGATTCTTTGTTTCAGTGTT |

In some embodiments, the plurality of pre-determined alleles used to determine a risk score for opioid use disorder and/or relapse comprise at least one allele selected from the group consisting of: chr11:113399438 of gene ANKK1; chr11:27643996 of gene BDNFOS/antiBDNF; chr1:224706393 of gene CNIH3; chr6:88150763 of gene CNR1; chr16:3745362 of gene CREBBP; chr22:38287631 of gene CSNK1E; chr11:113425897 of gene DRD2; chr11:113441417 of gene DRD2; chr11:113426463 of gene DRD2; chr11:113414814 of gene DRD2; chr11:113412966 of gene DRD2; chr11:113425564 of gene DRD2; chr3:114162776 of gene DRD3; chr3:114140326 of gene DRD3; chr11:636784 of gene DRD4; chr15:26774621 of gene GABRB3; chr19:1005231 of gene GABRB3; chr1:163535374 of gene intergenic g.163535374G; chr1:28855013 of gene OPRD1; chr1:28863085 of gene OPRD1; chr6:154040884 of gene OPRM1; chr8:56447926 of gene PENK; chr5:1446274 of gene SLC6A3; chr2:75198602 of gene TACR1; chr2:75135918 of gene TACR1; chr4:103643921 of gene TACR3; chr4:103585232 of gene TACR3; chr1:68194522 of gene WLS; chr2:184668853 of gene ZNF804A; and chr2:184913701 of gene ZNF804A. The sequences for this listed plurality of pre-determined alleles are provided in Table 25.

In some embodiments, the plurality of pre-determined alleles used to determine a risk score for opioid use disorder and/or relapse comprise at least one allele selected from the group consisting of: chr6:154039662 of gene OPRM1 118A>G; chr19:41006936 of gene CYP2B6*13*6*7*9+516G>T; chr22:42130692 of gene CYP2D6*4*10*1 4A+100C>T; chr1:11796321 of gene MTHFR 677C>T; CYP2C9 non EM (IM or PM); and chr7:99768693 of gene CYP3A4*22 intron6 15389C>T.

### Opioid Use Disorder Scoring

In some embodiments, a method for assessing whether a subject is at risk of opioid use disorder is provided. The method comprises: (1) obtaining a biological sample from a subject; (2) performing an allelic analysis on the biological sample to determine the presence of a plurality of pre-determined alleles, wherein the plurality of pre-determined alleles comprise one or more genomic targets selected from Table 1; (3) assigning a count for each of the alleles in the plurality of pre-determined alleles that was present in the biological sample; (4) determining a risk score based upon a total count using a SNP Model, wherein the risk score identifies a severity of the genetic predisposition to opioid addiction; and (5) administering a medical assisted treatment procedure based on the risk score identified in the subject.

In other embodiments, a method for assessing whether a subject is at risk of opioid use disorder is provided. The method comprises: (1) obtaining a biological sample from a subject; (2) performing an allelic analysis on the biological sample to determine the presence of a plurality of pre-determined alleles, wherein the plurality of pre-determined alleles comprise two or more genomic targets selected from Table 1; (3) assigning a count for each of the alleles in the plurality of pre-determined alleles that was present in the biological sample; (4) determining a risk score based upon a total count using a SNP Model, wherein the risk score identifies a severity of the genetic predisposition to opioid addiction; and (5) administering a medical assisted treatment procedure based on the risk score identified in the subject.

In determining the scoring strategy for opioid use disorder using SNPs, a mutation allele or wild type allele could be a risk allele. In addition, some SNPs need a single copy of the risk allele to elevate the risk of OUD, while other SNPs need two copies of the risk allele to elevate the risk of OUD. The OUD risk score modeling process, as used herein, includes the following steps: Step 1) identify risk alleles and the number needed to express the risk; Step 2) develop one or more risk score models to predict OUD; Step 3) Choose an accurate model based on area under the receiver operating characteristic curve (AUROC); and Step 4) determine clinically reasonable threshold points.

Upon determination of an OUD risk score using a plurality of risk alleles selected from Table 1, a medical assisted treatment procedure or patient therapy may then be provided to the patient. Patients determined to be at higher risk of OUD could receive reduced quantities of opioids dispensed and/or receive increased monitoring/more frequent visits with a healthcare professional. Conversely, patients determined to be at a lower risk of OUD may require less frequent monitoring and may be appropriate for receiving larger quantities of opioids between prescription fills. Additionally, patients determined to be at a higher risk of relapse may have justification for longer inpatient rehabilitation stays and/or longer intensive outpatient rehabilitation support. Conversely, patients determined to be at a lower risk of relapse may justify an earlier transition from inpatient rehabilitation to intensive outpatient rehabilitation. There may be additional benefit for patients to understand their own risks so as to have a better appreciation for the genetic basis of disease and empowerment over their own treatment decisions.

### Step 1: Identifying Risk SNP and Allele

A set of logistic regressions was conducted to identify SNPs that are significantly associated with the diagnosis of an OUD. Each SNP was coded to have three levels: a) having two wild type copies, b) having one wild type copy and one mutation copy, and c) two mutation copies. Two sets of odds ratios (ORs) were calculated for each SNP. For the first odds ratio, the odds of having OUD between those with two wild type copies and those with one or more mutation copies were compared. For the second odds ratio, the odds of having OUD between those subjects having two mutation copies and those having one or more wild type copies were compared. An OR was determined to be significant if the p-value was 0.05 or less. Because the strength of association between certain SNPs and the OUD could be different between male and female, the analysis can be stratified by sex. As a result, 10 SNPs for female and 9 SNPs for male were identified as being significantly associated with OUD. A listing of those SNPs and their corresponding risk alleles are shown in Table 2 (female) and Table 3 (male).

TABLE 2
SNPs Significantly Associated with OUD in Females.

| | | | Risk | | Odds | |
|---|---|---|---|---|---|---|
| | | Risk | Geno- | | Ratio | |
| Assay Name | Gene Symbol | Allele | types | NCBI SNP | (OR) | p-value |
| BDNFOS/antiBDNF | BDNFOS/ | C | T/C, | rs11030096 | 2.2 | 0.016 |
| n305 + 3991T > C | antiBDNF | | C/T, or | | | |
| | | | C/C | | | |
| DRD2 c-31- | DRD2 | A | G/A, | rs1079596 | 1.9 | 0.040 |
| 1215G > A | | | A/G, or | | | |
| | | | A/A | | | |
| DRD2 c-31- | DRD2 | G | A/G, | rs1125394 | 1.9 | 0.040 |
| 1781A > G | | | G/A, or | | | |
| | | | G/G | | | |
| DRD3 c527- | DRD3 | C | T/C, | rs9288993 | 4.7 | 0.022 |
| 630T > C | | | C/T, or | | | |
| | | | C/C | | | |
| GABRB3 c- | GABRB3 | T | T/T | rs4906902 | 2.0 | 0.027 |
| 1659T > C | | | | | | |
| OPRM1 | OPRM1 | C | C/C | rs510769 | 1.7 | 0.044 |
| c290 + 1050C > T | | | | | | |
| TACR1 aka | TACR1 aka | T | T/T | rs735668 | 2.0 | 0.032 |
| NK1R c390- | NK1R | | | | | |
| 15150T > G | | | | | | |
| ZNF804A | ZNF804A | T | T/T | rs7597593 | 2.5 | 0.024 |
| c111 + 69783T > C | | | | | | |
| DRD3 | DRD3 | C | C/T, | rs2654754 | 3.3 | 0.030 |
| c723 + 2551C > T | | | T/C, or | | | |
| | | | C/C | | | |
| OPRM1 118A > G | OPRM1 | A | A/A | rs1799971 | 2.8 | 0.004 |

TABLE 3
SNPs Significantly Associated with OUD in Males.

| | | | Risk | | | |
|---|---|---|---|---|---|---|
| | | Risk | Geno- | | | |
| Assay Name | Gene Symbol | Allele | type | NCBI SNP | OR | p-value |
| CNR1 c-63- | CNR1 | A | A/A | rs2023239 | 2.0 | 0.029 |
| 5426A > G | | | | | | |
| TACR3 aka | TACR3 aka | G | A/G, | rs4530637 | 2.6 | 0.050 |
| NK3R | NK3R | | G/A, or | | | |
| g.103585232A > G | | | G/G | | | |
| TACR3 aka | TACR3 aka | C | C/T, | rs1384401 | 2.9 | 0.049 |
| NK3R | NK3R | | T/C, or | | | |
| c888 + 12273C > T | | | C/C | | | |
| EXOC4 c1183- | EXOC4 | T | T/T | rs718656 | 1.9 | 0.042 |
| 39230C > T | | | | | | |
| DRD3 c271- | DRD3 | T | T/C, | rs324029 | 2.0 | 0.035 |
| 2909T > C | | | C/T, or | | | |
| | | | T/T | | | |
| DRD3 c25G > A | DRD3 | G | G/A, | rs6280 | 2.5 | 0.006 |
| | | | A/G, or | | | |
| | | | G/G | | | |
| CNR1 c-63- | CNR1 | G | G/G | rs6928499 | 2.0 | 0.029 |
| 6233C > G | | | | | | |
| CYP2B6*13*6*7*9 + | CYP2B6 | G | G/G | rs3745274 | 2.0 | 0.034 |
| 516 G > T | | | | | | |
| CYP2D6*4*10*14A + | CYP2D6 | C | C/C | rs1065852 | 3.3 | 0.043 |
| 100C > T | | | | | | |
| not*4M | | | | | | |

In certain embodiments, the SNP Model may include determining a weighted algorithm based on the ORs of the 10 SNPs for female and 9 SNPs for male with p-values of 0.05 or less, as provided in Tables 2 and 3 above. The weighted algorithm may be further based on the genetic codes as well as clinical (phenotype) data utilizing a logistic regression separately between male and female SNPs.

In some embodiments, the plurality of pre-determined alleles comprise at least one allele selected from the group consisting of:

allele C+ of gene BDNFOS/antiBDNF (rs11030096) wherein C+ includes T/C, C/T, or C/C;

allele A+ of gene DRD2 (rs1079596) wherein G+ includes G/A, A/G, or A/A;

allele G+ of gene DRD2 (rs1125394) wherein G+ includes A/G, G/A, or G/G;

allele C+ of gene DRD3 (rs9288993) wherein C+ includes T/C, C/T, or C/C;

allele T/T of gene GABRB3 (rs4906902);

allele C/C of gene OPRM1 (rs510769);

allele T/T of gene TACR1 (rs735668);

allele T/T of gene ZNF804A (rs7597593);

allele C+ of gene DRD3 (rs2654754) wherein C+ includes C/T, T/C, or C/C; and/or allele A/A of gene OPRM1 (rs1799971).

In other embodiments, the plurality of pre-determined alleles comprise at least one allele selected from the group consisting of:

allele A/A of gene CNR1 (rs2023239);

allele G+ of gene TACR3 (rs4530637) wherein G+ includes A/G, G/A, or G/G;

allele C+ of gene TACR3 (rs1384401) wherein C+ includes C/T, T/C, or C/C;

allele T/T of gene EXOC4 (rs718656);

allele T+ of gene DRD3 (rs324029) wherein T+ includes T/C, C/T, or T/T;

allele G+ of gene DRD3 (rs6280) wherein G+ includes G/A, A/G, or G/G;

allele G/G of gene CNR1 (rs6928499);

allele G/G of gene CYPB6 (rs3745274); and/or

allele C/C of gene CYP2D6 (rs1065852).

In further embodiments, the plurality of pre-determined alleles comprise at least one allele selected from the group consisting of:

allele C/C of gene CNIH3 (rs1369846);

allele A/A of gene CNIH3 (rs1436171);

allele A/A of gene GRIN3A (rs17189632);

allele C+ of gene HTR3B (rs11606194) wherein C+ includes T/C, C/T, or C/C;

allele C/C of gene OPRD1 (rs2234918);

allele G/G of gene WLS (rs1036066);

allele G+ of gene intergenic (rs965972) wherein G+ includes G/A, A/G, or G/G;

allele C/C of gene MTHFR (rs1801133); and/or

allele G/G of gene MTHFR (rs1801133).

In some embodiments, the plurality of pre-determined alleles comprise at least one allele selected from the group consisting of:

allele T/T of gene DRD3 (rs9825563);

allele T/T of gene GAL (rs948854);

allele C+ of gene NR4A2 (rs1405735) wherein C+ includes C/G, G/C, or C/C;

allele A+ of gene OPRM (rs9479757) wherein A+ includes G/A, A/G, or A/A; and/or allele T+(A+) of gene CYP3A4 (rs35599367) wherein T+ includes C/T, T/C, or T/T and wherein A+ includes G/A, A/G, or A/A.

### Step 2: OUD Risk Score Modeling

Model 1: Sex-stratified, count SNPs with appropriate number of risk allele copies. The OUD risk score was calculated as the sum of SNPs that had the risk alleles as identified above in Tables 2 and 3. For example, EXOC4 was not counted towards the risk score if the subject had C/T, because two copies of T are required in order it to be counted. Similarly, DRD3(rs6280) was counted only once if a subject had at least one copy of G, regardless of the number of copies. Female subjects can have a risk score ranging from 0 to 10 and male subjects can have a risk score ranging from 0 to 9. Table 4 shows the distribution of risk scores by OUD in male and female subjects.

TABLE 4
Risk score distribution by OUD in Model 1.

| | Female | | | Male | | |
|---|---|---|---|---|---|---|
| Risk score | OUD No | OUD Yes | Total | OUD No | OUD Yes | Total |
| 0 | 3 | 0 | 3 | 0 | 0 | 0 |
| 1 | 7 | 1 | 8 | 1 | 0 | 1 |
| 2 | 26 | 9 | 35 | 6 | 1 | 7 |
| 3 | 37 | 15 | 52 | 3 | 2 | 5 |
| 4 | 28 | 21 | 49 | 6 | 10 | 16 |
| 5 | 11 | 24 | 35 | 22 | 11 | 33 |
| 6 | 6 | 15 | 21 | 17 | 23 | 40 |
| 7 | 1 | 6 | 7 | 13 | 23 | 36 |
| 8 | 0 | 1 | 1 | 6 | 22 | 28 |
| 9 | 0 | 1 | 1 | 2 | 15 | 17 |
| Total | 119 | 93 | 212 | 76 | 107 | 183 |

Model 2: Sex-stratified, count risk alleles, maximum 2 per SNP). In some embodiments, the OUD risk score was calculated as the sum of risk alleles. For example, if a subject had “C/T” for EXOC4, 1 was added towards the risk score. In other examples, if a subject had “T/T” for EXOC4, 2 was added towards the risk score because two risk alleles were present. Accordingly, with the possibility of having a maximum count of 2 per SNP, female subjects can have a risk score ranging from 0 to 20 and male subjects can have a risk score ranging from 0 to 18. The distribution of risk scores by OUD generated using SNP Model 2 are provided in Table 5.

TABLE 5
OUD Risk Score Distribution using Model 2.

| | Female | | | Male | | |
|---|---|---|---|---|---|---|
| Risk score | OUD No | OUD Yes | Total | OUD No | OUD Yes | Total |
| 3 | 0 | 1 | 1 | 0 | 0 | 0 |
| 4 | 6 | 2 | 8 | 0 | 0 | 0 |
| 5 | 9 | 0 | 9 | 0 | 0 | 0 |
| 6 | 12 | 6 | 18 | 2 | 0 | 2 |
| 7 | 30 | 11 | 41 | 5 | 1 | 6 |
| 8 | 28 | 14 | 42 | 6 | 2 | 8 |
| 9 | 15 | 26 | 41 | 3 | 5 | 8 |
| 10 | 13 | 11 | 24 | 11 | 7 | 18 |
| 11 | 1 | 13 | 14 | 12 | 14 | 26 |
| 12 | 4 | 7 | 11 | 9 | 16 | 25 |
| 13 | 1 | 1 | 2 | 17 | 21 | 38 |
| 14 | 0 | 0 | 0 | 6 | 22 | 28 |
| 15 | 0 | 1 | 1 | 3 | 14 | 17 |
| 16 | 0 | 0 | 0 | 2 | 4 | 6 |
| 17 | 0 | 0 | 0 | 0 | 1 | 1 |

Model 3: Non-stratified, count SNPs with appropriate number of risk allele copies. Model 3 calculates the risk score in the same way as Model 1, but it does not stratify by sex. Both male and female subjects can accordingly have a risk score ranging from 0 to 19 regardless of their sex/gender. Table 6 provides the distribution of risk scores by OUD in SNP Model 3.

TABLE 6
Risk score distribution by OUD in Model 3.

| Risk score | OUD No | OUD Yes | Total |
|---|---|---|---|
| 3 | 1 | 0 | 1 |
| 4 | 1 | 1 | 2 |
| 5 | 4 | 1 | 5 |
| 6 | 18 | 6 | 24 |
| 7 | 28 | 10 | 38 |
| 8 | 30 | 28 | 58 |
| 9 | 35 | 33 | 68 |
| 10 | 34 | 33 | 67 |
| 11 | 23 | 30 | 53 |
| 12 | 12 | 26 | 38 |
| 13 | 8 | 22 | 30 |
| 14 | 1 | 6 | 7 |
| 15 | 0 | 3 | 3 |
| 16 | 0 | 1 | 1 |
| Total | 195 | 200 | 395 |

In some embodiments, determining the risk score based upon summing the plurality of counts could include the SNP Model 1: Sex-stratified, count SNPs with appropriate number of risk allele copies approach (sex-stratified single count SNP model). In other embodiments, determining the risk score based upon summing the plurality of counts could include the SNP Model 2: Sex-stratified, count risk alleles, maximum 2 per SNP) approach (sex-stratified double count SNP model). In still other embodiments, determining the risk score based upon summing the plurality of counts could include the SNP Model 3: Non-stratified, count SNPs with appropriate number of risk allele copies approach (non-sex-stratified single count SNP model).

### Step 3: Model Validation.

A receiver operating characteristic (ROC) curve is a performance measurement for classification at multiple threshold levels. The area under the ROC curve (AUROC) is particularly useful to measure the discrimination (or accuracy), which is the ability of the risk score model to correctly classify those with and without OUD. The AUROC takes values from 0 to 1, where a value of 0 indicates a perfectly inaccurate test and a value of 1 reflects a perfectly accurate test. In general, an AUROC of 0.5 indicates no discrimination, 0.7-0.8 is considered acceptable, 0.8 to 0.9 is considered excellent, and more than 0.9 is considered outstanding. Table 7 lists the AUROC results of the three models (SNP Model 1, SNP Model 2, and SNP Model 3) discussed in Step 2 above.

TABLE 7
Area under the ROC curve

| | AUROC | | |
|---|---|---|---|
| | Female | Male | All |
| Model 1 | 0.9003 | 0.8831 | n/a |
| Model 2 | 0.8885 | 0.8790 | n/a |
| Model 3 | n/a | n/a | 0.8735 |

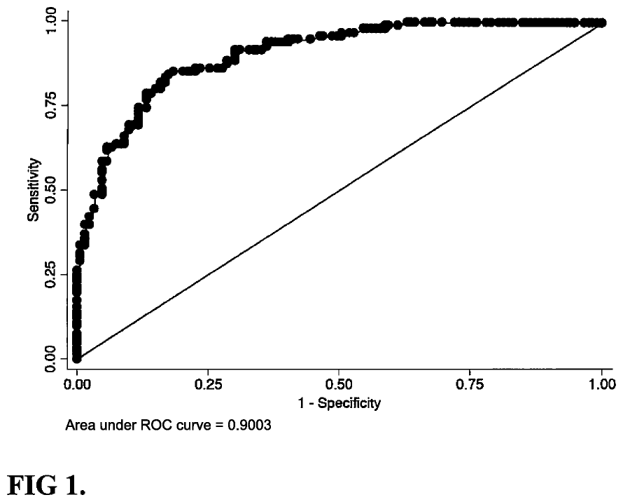

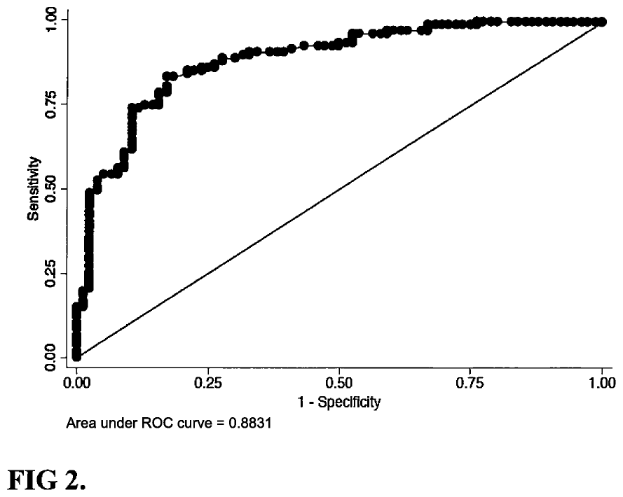

The results provided in Table 7 suggest that Model 1 demonstrated excellent accuracy for both female and male subjects. FIG. 1 and FIG. 2 plot the ROC curves produced using Model 1. In some embodiments, the AUROC value may range from about 0.6 to about 1.0, from about 0.7 to about 1.0, from about 0.8 to about 1.0, from about 0.9 to about 1.0, from about 0.6 to about 0.7, from about 0.6 to about 0.8, from about 0.6 to about 0.9, from about 0.7 to about 0.8, from about 0.7 to about 0.9, from about 0.7 to about 1.0, from about 0.8 to about 0.9, from about 0.8 to about 1.0, or from about 0.9 to about 1.0. These provided AUROC values may be calculated or determined using Model 1, Model 2, Model 3, or any combinations thereof.

### Step 4: Cut-Off Analysis

Based on the AUROC, SNP Model 1 was identified as being very accurate. Accordingly, the optimal cut-off (threshold) point of the risk score in Model 1 was then tested. Sensitivity, specificity, positive predictive value (PPV) and negative predictive value (NPV) were calculated for all possible threshold levels and shown in Table 8 (female) and Table 9 (male). It was estimated that the threshold of risk score 5 for female and 6 for male would maximize the sum of sensitivity and specificity. The sensitivity (“sen”), specificity (“spec”), positive predictive value and negative predictive value are shown in Tables 8 and 9. However, it should be noted that the trade-off between sensitivity and specificity should be considered for each clinical condition. It is possible that choosing a threshold that maximizes the sensitivity while losing some specificity may be more beneficial in some cases (i.e., cancer).

The levels of risk with respect to a subject having a genomic susceptibility to opioid use disorder were determined and assigned using the following rules: (1) the level of risk is “low” if the negative predictive value (NPV) is greater than 80%; (2) the level of risk is “moderate” if the NPV is greater than 65% and less than 80%; (3) the level of risk is “high” if the positive predictive value (PPV) is greater than 65%; and (4) the level of risk is “very high” if the PPV is greater than 80%.

The risk scoring system using Model 1 to evaluate the 10 SNPs provided in Table 2 for females includes different levels of risk based on the subject's corresponding risk score. Referring to Table 8, in some embodiments, a female having a risk score less than 3 corresponds to a low chance of OUD; a risk score greater than or equal to 3 and less than 5 corresponds to a moderate chance of OUD; a risk score greater than or equal to 5 and less than or equal to 7 corresponds to a high chance of OUD; and a risk score greater than or equal to 7 corresponds to a very high chance of OUD.

TABLE 8
Test Validation Estimates in Model 1 Female.

| Risk score | | | | | Sen + | Level of |
|---|---|---|---|---|---|---|
| threshold | Sensitivity | Specificity | PPV | NPV | Spec | Risk |
| 2 | 99% | 8% | 46% | 91% | 107% | low |
| 3 | 89% | 30% | 50% | 78% | 119% | moderate |
| 4 | 73% | 61% | 60% | 74% | 134% | moderate |
| 5 | 51% | 85% | 72% | 69% | 135% | high |
| 6 | 25% | 94% | 77% | 62% | 119% | high |
| 7 | 9% | 99% | 89% | 58% | 108% | very high |

The risk scoring system using Model 1 to evaluate the 9 SNPs provided in Table 3 for males includes different levels of risk based on the subject's corresponding risk score. Referring to Table 9, in some embodiments, a male having a risk score less than 4 corresponds to a low chance of OUD; a risk score greater than or equal to 4 and less than 6 corresponds to a moderate chance of OUD; a risk score greater than or equal to 6 and less than or equal to 8 corresponds to a high chance of OUD; and a risk score greater than or equal to 8 corresponds to a very high chance of OUD.

TABLE 9
Test Validation Estimates in Model 1 Male.

| Risk score | | | | | Sen + | Level of |
|---|---|---|---|---|---|---|
| threshold | Sensitivity | Specificity | PPV | NPV | spec | Risk |
| 2 | 100% | 1% | 59% | 100% | 101% | low |
| 3 | 99% | 9% | 61% | 88% | 108% | low |
| 4 | 97% | 13% | 61% | 77% | 110% | moderate |
| 5 | 88% | 21% | 61% | 55% | 109% | moderate |
| 6 | 78% | 50% | 69% | 61% | 128% | high |
| 7 | 56% | 72% | 74% | 54% | 128% | high |
| 8 | 35% | 89% | 82% | 49% | 124% | very high |

Generally, as thresholds rise, specificity and PPV also rise, but sensitivity falls. In some embodiments, higher sensitivity can be realized by lowering the threshold, albeit at the cost of lower specificity and PPV. Conversely, if higher PPV is required, it can often be realized by raising the threshold, albeit at the cost of lower sensitivity.

The levels of risk with respect to a subject having a genomic susceptibility to opioid use disorder were determined and assigned using the following rules: (1) the level of risk is “low” if the negative predictive value (NPV) is greater than 80%; (2) the level of risk is “moderate” if the NPV is greater than 65% and less than 80%; (3) the level of risk is “high” if the positive predictive value (PPV) is greater than 65%; and (4) the level of risk is “very high” if the PPV is greater than 80%.

The risk scoring system using SNP Model 2 to evaluate the 10 SNPs provided in Table 2 for females includes different levels of risk based on the subject's corresponding risk score. Referring to Table 10, in some embodiments, a female having a risk score less than 7 corresponds to a low chance of OUD; a risk score greater than or equal to 7 and less than 10 corresponds to a moderate chance of OUD; a risk score greater than or equal to 11 and less than 14 corresponds to a high chance of OUD; and a risk score greater than or equal to 14 corresponds to a very high chance of OUD.

TABLE 10
Test Validation Estimates in Model 2 Female

| Risk score | | | | | |
|---|---|---|---|---|---|
| threshold | Sensitivity | Specificity | PPV | NPV | Sen + spec |
| 6 | 97% | 13% | 46% | 83% | 110% |
| 7 | 90% | 23% | 48% | 75% | 113% |
| 8 | 78% | 48% | 54% | 74% | 126% |
| 9 | 63% | 71% | 63% | 71% | 134% |
| 10 | 35% | 84% | 63% | 63% | 119% |
| 11 | 24% | 95% | 79% | 61% | 119% |
| 12 | 10% | 96% | 64% | 58% | 106% |
| 13 | 2% | 99% | 67% | 56% | 101% |
| 14 | 1% | 100% | 100% | 56% | 101% |
| 15 | 1% | 100% | 100% | 56% | 101% |

The risk scoring system using Model 2 to evaluate the 9 SNPs provided in Table 3 for males includes different levels of risk based on the subject's corresponding risk score. Referring to Table 11, in some embodiments, a male having a risk score less than 10 corresponds to a low chance of OUD; a risk score equal to 10 corresponds to a moderate chance of OUD; a risk score greater than or equal to 11 corresponds to a high chance of OUD.

TABLE 11
Test Validation estimates in Model 2 Male

| Risk score | | | | | |
|---|---|---|---|---|---|
| threshold | Sensitivity | Specificity | PPV | NPV | Sen + spec |
| 7 | 100% | 3% | 59% | 100% | 103% |
| 8 | 99% | 9% | 61% | 88% | 108% |
| 9 | 97% | 17% | 62% | 81% | 114% |
| 10 | 93% | 21% | 62% | 67% | 114% |
| 11 | 86% | 36% | 65% | 64% | 122% |
| 12 | 73% | 51% | 68% | 57% | 124% |
| 13 | 58% | 63% | 69% | 52% | 121% |
| 14 | 38% | 86% | 79% | 50% | 124% |
| 15 | 18% | 93% | 79% | 45% | 111% |
| 16 | 5% | 97% | 71% | 42% | 102% |

The risk scoring system using Model 3 to evaluate the 19 SNPs provided in Tables 2 and 3 for both females and males includes different levels of risk based on the subject's corresponding risk score. Referring to Table 12, in some embodiments, a subject having a risk score less than 5 corresponds to a low chance of OUD; a risk score greater than or equal to 5 or less than 11 corresponds to a moderate chance of OUD; a risk score greater than or equal to 11 and less than 14 corresponds to a high chance of OUD; and a risk score greater than or equal to 14 corresponds to a very high chance of OUD.

TABLE 12
Test Validation Estimates in Model 3

| Risk score | | | | | |
|---|---|---|---|---|---|
| threshold | Sensitivity | Specificity | PPV | NPV | Sen + spec |
| 4 | 100% | 1% | 51% | 100% | 101% |
| 5 | 100% | 1% | 51% | 67% | 101% |
| 6 | 99% | 3% | 51% | 75% | 102% |
| 7 | 96% | 12% | 53% | 75% | 108% |
| 8 | 91% | 27% | 56% | 74% | 118% |
| 9 | 77% | 42% | 58% | 64% | 119% |
| 10 | 61% | 60% | 61% | 60% | 121% |
| 11 | 44% | 77% | 67% | 57% | 121% |
| 12 | 29% | 89% | 73% | 55% | 118% |
| 13 | 16% | 95% | 78% | 53% | 111% |
| 14 | 5% | 99% | 91% | 51% | 104% |
| 15 | 2% | 100% | 100% | 50% | 102% |

### Relapse Risk Scoring

In some embodiments, a method for assessing whether a subject is at risk of opioid relapse is provided. The method comprises: (1) obtaining a biological sample from a subject; (2) performing an allelic analysis on the biological sample to determine the presence of a plurality of pre-determined alleles, wherein the plurality of pre-determined alleles comprise two or more genomic targets selected from Table 1; (3) assigning a count for each of the alleles in the plurality of pre-determined alleles that was present in the biological sample; (4) determining a risk score based upon a total count using a SNP Model, wherein the risk score identifies a severity of the genetic predisposition to opioid addiction; and (5) administering a medical assisted treatment procedure based on the risk score identified in the subject.

In some embodiments, a method of obtaining and utilizing a relapse risk score for assessing a genetic predisposition to addiction relapse is provided. The method comprises: (1) obtaining a biological sample from a subject; (2) performing an allelic analysis on the biological sample to determine the presence of a plurality of pre-determined alleles by assigning a plurality of counts, wherein the plurality of pre-determined alleles comprise one or more genomic targets selected from Table 1; (3) determining a risk score based upon summing the plurality of counts; (4) comparing the risk score with one or more predetermined reference values, wherein the subject is determined to have a risk level of opioid addiction if the risk score is greater than a threshold value using a SNP model; and (5) administering a medical assisted treatment procedure based on the risk score identified in the subject.

Upon determination of an opioid relapse risk score using a plurality of risk alleles selected from Table 1, a medical assisted treatment procedure or patient therapy may then be provided to the patient. Patients determined to be at higher risk of relapse could receive reduced quantities of opioids dispensed and/or receive increased monitoring/more frequent visits. Conversely, patients determined to be at a lower risk of relapse may require less frequent monitoring and may be appropriate for receiving larger quantities of opioids between prescription fills. Additionally, patients determined to be at a higher risk of relapse may have justification for longer inpatient rehabilitation stays and/or longer intensive outpatient rehabilitation support. Conversely, patients determined to be at a lower risk of relapse may justify an earlier transition from inpatient rehabilitation to intensive outpatient rehabilitation. There may be additional benefit for patients to understand their own risks so as to have a better appreciation for the genetic basis of disease and empowerment over their own treatment decisions.

In developing the scoring technique to determine OUD relapse risk, some SNPs need a single copy of the risk allele to elevate the risk of OUD relapse, while other SNPs need two copies of the risk allele to elevate the risk of OUD relapse. The OUD relapse risk score modeling process, as used herein, includes the following steps: Step 1) identify risk alleles and the number needed to express the risk; Step 2) develop one or more risk score models to predict OUD relapse; Step 3) Choose an accurate model based on area under the receiver operating characteristic curve (AUROC); and Step 4) determine clinically reasonable threshold points.

In developing the scoring technique to determine opioid relapse risk, the following factors may be considered including that a mutation type allele or wild type allele could be a risk allele.

### Step 1: Identifying Risk SNP and Allele

A set of logistic regression was conducted to identify SNPs that are significantly associated with OUD relapse among persons receiving a buprenorphine-naloxone combination as a medication-assisted treatment (MAT). Each SNP was coded to have three levels: a) having two wild type copies, b) having one wild type copy and one mutation copy, and c) two mutation copies. Two odds ratios (ORs) were calculated for each SNP. First, the odds of having OUD between those with two wild type copies and those with one or more mutation copies were compared. Second, the odds of having OUD between those with two mutation copies and those with one or more wild type copies were compared. An OR was determined as significant if the p-value was 0.05 or less. Because the strength of association between certain SNPs and relapse could be different between male and female, the analysis was stratified by sex. As a result, 9 SNPs/phenotype for female and 6 SNPs/phenotype for male were identified as being significantly associated with relapse. Two SNPs were identified as significantly associated with relapse in the group as a whole, however those were not significant in a stratified group (potentially due to smaller sample size). A listing of those SNPs and their corresponding risk alleles are shown in Tables 13-15.

TABLE 13
SNPs Significantly Associated with Opioid Relapse in Females.

| | | | Risk | | | |
|---|---|---|---|---|---|---|
| | Gene | Risk | Geno- | | | |
| Assay name | Symbol | Allele | type | NCBI SNP | OR | P-value |
| CNIH3 c198 + 21550C > T | CNIH3 | C | C/C | rs1369846 | 3.85 | 0.036 |
| CNIH3 c198 + 9283A > G | CNIH3 | A | A/A | rs1436171 | 3.7 | 0.033 |
| GRIN3A c2766 + 7656A > T | GRIN3A | A | A/A | rs17189632 | 4.17 | 0.037 |
| HTR3B c213 + 804T > C | HTR3B | C | T/C, | rs11606194 | 6.45 | 0.011 |
| | | | C/T, | | | |
| | | | or C/C | | | |
| OPRD1 c921C > T | OPRD1 | C | C/C | rs2234918 | 3.7 | 0.033 |
| WLS c107-301T > G | WLS | G | G/G | rs1036066 | 6.67 | 0.018 |
| intergenic g.193494720G > A | intergenic | G | G/A, | rs965972 | 7.69 | 0.029 |
| | | | A/G, | | | |
| | | | or G/G | | | |
| MTHFR 677C > T | MTHFR | C/C | C/C | rs1801133 | 6.25 | 0.009 |
| | | (G/G) | (G/G) | | | |
| CYP2C9 non EM (IM or | | | | | 3.36 | 0.043 |
| PM) | | | | | | |

TABLE 14
SNPs Significantly Associated with Opioid Relapse in Males.

| | | | Risk | | | |
|---|---|---|---|---|---|---|
| | Gene | Risk | Geno- | | | |
| Assay name | Symbol | Allele | type | NCBI SNP | OR | P-value |
| DRD3 c-155-2597T > C | DRD3 | T | T/T | rs9825563 | 5.13 | 0.019 |
| GAL c-1998C > T | GAL | T | T/T | rs948854 | 4.01 | 0.046 |
| NR4A2 g.156377320C > G | NR4A2 | C | C/G, | rs1405735 | 3.57 | 0.033 |
| | | | G/C, or | | | |
| | | | C/C | | | |
| OPRM c643 + 31G > A | OPRM | A | G/A, | rs9479757 | 3.82 | 0.034 |
| | | | A/G, or | | | |
| | | | A/A | | | |
| CYP3A4*22 intron6 | CYP3A4 | T (A) | C/T | rs35599367 | 13 | 0.006 |
| 15389C > T | | | (G/A), | | | |
| | | | T/C | | | |
| | | | (A/G), | | | |
| | | | or T/T | | | |
| | | | (A/A) | | | |
| CYP 3A4 IM | | | | | 13 | 0.006 |

In male subjects, CYP3A4*22 intron6 15389C>T (rs35599367) predicted the CYP3A4 phenotype perfectly. As a result, the estimate has the same odds ratio and p-value. This was not the case in female subjects.

TABLE 15
SNPs that are significantly associated with
relapse in a male and female combined group.

| | | | Risk | | | |
|---|---|---|---|---|---|---|
| | Gene | Risk | Geno- | | | |
| Assay name | Symbol | Allele | type | NCBI SNP | OR | P-value |
| DRD3 c25G > A | DRD3 | A | A/A | rs6280 | 3 | 0.009 |
| SORCS3 c628-28349A > G | SORCS3 | G | A/G, | rs728453 | 3.06 | 0.018 |
| | | | G/A, | | | |
| | | | or G/G | | | |

In certain embodiments, the SNP Model may include determining a weighted algorithm based on the ORs of the 9 SNPs/phenotype for female, 6 SNPs/phenotype for male, 2 SNPs for both sexes with p-values of 0.05 or less, as provided in Tables 13-15 above. The weighted algorithm may be further based on the genetic codes as well as clinical (phenotype) data utilizing a logistic regression separately between male and female SNPs.

### Step 2: Relapse Risk Score Modeling

Model 1: Sex-stratified, count SNPs with appropriate number of risk allele copies. The OUD relapse risk score was calculated as the sum of SNPs that had the risk alleles as identified above (Tables 13, 14, and 15). For example, GAL (rs948854) was not counted towards the risk score if a subject has C/T, because two copies of T are required in order it to be counted. Similarly, OPRM (rs9479757) was counted only once if a subject had at least one copy of A, regardless of the number of copies. For CYP2C9 phenotype, if a subject was not an EM, 1 was added towards the risk score. Also, two SNPs that are significantly associated with relapse in a male and female combined group (Table 15) were used in calculating risk scores for each of male and female. Female subjects can have a risk score ranging from 0 to 11 and male subjects can have a risk score ranging from 0 to 7. Table 16 shows the distribution of risk scores by relapse in male and female.

TABLE 16
Risk score distribution by relapse in Model 1.

| | Female | | | Male | | |
|---|---|---|---|---|---|---|
| Risk | Relapse | Relapse | | Relapse | Relapse | |
| score | No | Yes | Total | No | Yes | Total |
| 0 | 4 | 0 | 4 | 7 | 0 | 7 |
| 1 | 10 | 0 | 10 | 20 | 0 | 20 |
| 2 | 15 | 0 | 15 | 23 | 2 | 25 |
| 3 | 12 | 2 | 14 | 14 | 3 | 17 |
| 4 | 6 | 6 | 12 | 3 | 7 | 10 |
| 5 | 0 | 3 | 3 | 0 | 1 | 1 |
| 6 | 0 | 2 | 2 | 0 | 1 | 1 |
| 7 | 0 | 1 | 1 | | | |
| 8 | 0 | 1 | 1 | | | |
| 9 | 0 | 0 | 0 | | | |
| 10 | 0 | 1 | 1 | | | |
| 11 | 0 | 0 | 0 | | | |
| Total | 47 | 16 | 63 | 67 | 14 | 81 |

Model 2: Sex-stratified, count risk alleles, maximum 2 per SNP. The OUD relapse risk score was calculated as the sum of risk alleles. For example, if a subject had “G/A” for SORCS3 (rs728453), 1 was added towards the risk score. On the other hand, if a subject had “G/G” for SORCS3, 2 was added towards the risk score because two risk alleles were present. For CYP2C9 phenotype, if a subject was not an EM, 1 was added towards the risk score. Female subjects can have a risk score ranging from 0 to 21 and male subjects can have a risk score ranging from 0 to 13. The distribution of risk scores by relapse shown in Table 17.

TABLE 17
Risk score distribution by relapse in Model 2.

| | Female | | | Male | | |
|---|---|---|---|---|---|---|
| Risk | Relapse | Relapse | | Relapse | Relapse | |
| score | No | Yes | Total | No | Yes | Total |
| 0 | 0 | 0 | 0 | 0 | 0 | 0 |
| 1 | 0 | 0 | 0 | 1 | 0 | 1 |
| 2 | 1 | 0 | 1 | 2 | 0 | 2 |
| 3 | 2 | 0 | 2 | 10 | 0 | 10 |
| 4 | 1 | 0 | 1 | 16 | 0 | 16 |
| 5 | 3 | 1 | 4 | 19 | 3 | 22 |
| 6 | 9 | 1 | 10 | 16 | 2 | 18 |
| 7 | 7 | 1 | 8 | 2 | 7 | 9 |
| 8 | 8 | 1 | 9 | 1 | 1 | 2 |
| 9 | 9 | 1 | 10 | 0 | 1 | 1 |
| 10 | 4 | 2 | 6 | | | |
| 11 | 3 | 4 | 7 | | | |
| 12 | 0 | 1 | 1 | | | |
| 13 | 0 | 1 | 1 | | | |
| 14 | 0 | 1 | 1 | | | |
| 15 | 0 | 1 | 1 | | | |
| 16 | 0 | 0 | 0 | | | |
| 17 | 0 | 1 | 1 | | | |
| Total | 47 | 16 | 63 | 67 | 14 | 81 |

Model 3: Non-stratified, count SNPs with appropriate number of risk allele copies. Model 3 calculates the risk score in the same way as Model 1, but it does not stratify by sex. Subjects can have a risk score ranging from 0 to 16 regardless of sex. Table 18 shows the distribution of risk scores by relapse in Model 3.

TABLE 18
Risk score distribution by relapse in Model 3.

| Risk score | Relapse No | Relapse Yes | Total |
|---|---|---|---|
| 0 | 1 | 0 | 1 |
| 1 | 9 | 0 | 9 |
| 2 | 16 | 0 | 16 |
| 3 | 22 | 1 | 23 |
| 4 | 34 | 1 | 35 |
| 5 | 14 | 14 | 28 |
| 6 | 11 | 3 | 14 |
| 7 | 7 | 6 | 13 |
| 8 | 0 | 1 | 1 |
| 9 | 0 | 2 | 2 |
| 10 | 0 | 0 | 0 |
| 11 | 0 | 1 | 1 |
| 12 | 0 | 1 | 1 |
| Total | 114 | 30 | 144 |

In some embodiments, determining the risk score based upon summing the plurality of counts could include the SNP Model 1: Sex-stratified, count SNPs with appropriate number of risk allele copies approach (sex-stratified single count SNP model). In other embodiments, determining the risk score based upon summing the plurality of counts could include the SNP Model 2: Sex-stratified, count risk alleles, maximum 2 per SNP approach (sex-stratified double count SNP model). In still other embodiments, determining the risk score based upon summing the plurality of counts could include the SNP Model 3: Non-stratified, count SNPs with appropriate number of risk allele copies approach (non-sex-stratified single count SNP model).

### Step 3: Model Validation.

Receiver operating characteristic (ROC) curve is a performance measurement for classification at multiple threshold levels. The area under the ROC curve (AUROC) is particularly useful to measure the discrimination (or accuracy), which is the ability of the risk score model to correctly classify those with and without OUD. The AUROC takes values from 0 to 1, where a value of 0 indicates a perfectly inaccurate test and a value of 1 reflects a perfectly accurate test. In general, an AUROC of 0.5 indicates no discrimination, 0.7-0.8 is considered acceptable, 0.8 to 0.9 is considered excellent, and more than 0.9 is considered outstanding. Table 19 shows the AUROC results of the three models (SNP Model 1, SNP Model 2, and SNP Model 3) developed in Step 2.

TABLE 19
Area under the ROC curve

| | AUROC | | |
|---|---|---|---|
| | Female | Male | All |
| Model 1 | 0.9621 | 0.8971 | n/a |
| Model 2 | 0.8025 | 0.8737 | n/a |
| Model 3 | n/a | n/a | 0.7266 |

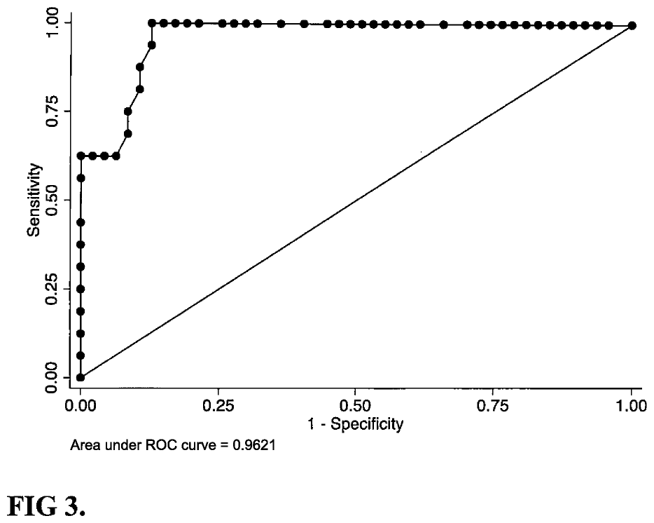

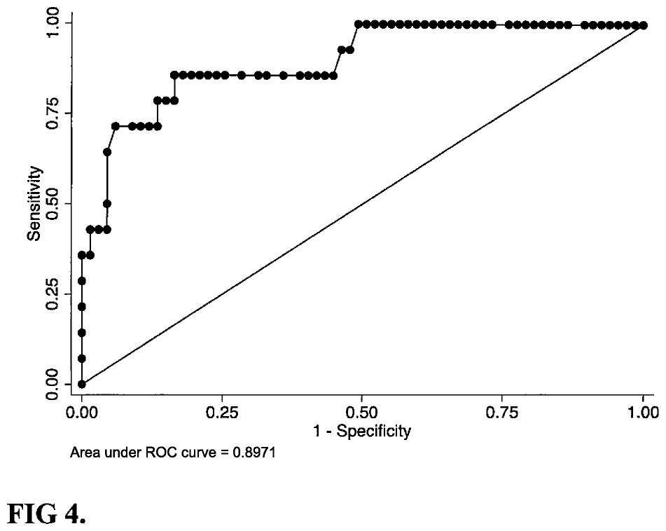

The results provided in Table 19 suggest that Model 1 demonstrated excellent accuracy for both female and male. FIG. 3 and FIG. 4 show the ROC curves from Model 1. In some embodiments, the AUROC value may range from about 0.6 to about 1.0, from about 0.7 to about 1.0, from about 0.8 to about 1.0, from about 0.9 to about 1.0, from about 0.6 to about 0.7, from about 0.6 to about 0.8, from about 0.6 to about 0.9, from about 0.7 to about 0.8, from about 0.7 to about 0.9, from about 0.7 to about 1.0, from about 0.8 to about 0.9, from about 0.8 to about 1.0, from about 0.9 to about 1.0. These provided AUROC values may be calculated or determined using Model 1, Model 2, Model 3, or any combinations thereof

### Step 4: Cut-Off Analysis

Based on the AUROC, SNP Model 1 was identified as an accurate means of analysis. Therefore, in the next step, the optimal cut-off (threshold) point of the risk score in Model 1 was tested. Sensitivity, specificity, positive predictive value (PPV) and negative predictive value (NPV) were calculated for all possible threshold levels and shown in Table 20 (female) and Table 21 (male). It was estimated that the threshold of risk score 4 for female and male would maximize the sum of sensitivity and specificity. However, it should be noted that the trade-off between sensitivity and specificity should be considered for each clinical condition. It is possible that choosing a threshold that maximizes the sensitivity while losing some specificity may be more beneficial in some cases (i.e., cancer). Generally, as thresholds rise, specificity and PPV also rise, but sensitivity falls. If higher sensitivity is desired, it can often be realized by lowering the threshold, albeit at the cost of lower specificity and PPV. Conversely, if higher PPV is required, it can often be realized by raising the threshold, albeit at the cost of lower sensitivity. Arbitrarily, we used the risk score threshold that generates the maximum sum of sensitivity and specificity as being associated with a moderate risk of relapse (yellow flag).

The risk scoring system using Model 1 to evaluate the 9 SNPs provided in Table 13 and 2 SNPs provided in Table 15 for females includes different levels of risk based on the subject's corresponding risk score. Referring to Table 20, in some embodiments, a female having a risk score less than 4 corresponds to a low chance of relapse; a risk score equal to 4 corresponds to a moderate chance of relapse; and a risk score greater than 4 corresponds to a high chance of relapse.

TABLE 20
Test validation estimates in Model 1 female.

| Risk score | | | | | |
|---|---|---|---|---|---|
| threshold | Sensitivity | Specificity | PPV | NPV | Sen + spec |
| 2 | 100% | 30% | 33% | 100% | 130% |
| 3 | 100% | 62% | 47% | 100% | 162% |
| 4 | 88% | 87% | 70% | 95% | 175% |
| 5 | 50% | 100% | 100% | 85% | 150% |
| 6 | 31% | 100% | 100% | 81% | 131% |
| 7 | 19% | 100% | 100% | 78% | 119% |
| 8 | 13% | 100% | 100% | 77% | 113% |
| 9 | 6% | 100% | 100% | 76% | 106% |
| 10 | 6% | 100% | 100% | 76% | 106% |

The risk scoring system using Model 1 to evaluate the 5 SNPs provided in Table 14 and 2 SNPs provided in Table 15 for males includes different levels of risk based on the subject's corresponding risk score. Referring to Table 21, in some embodiments, a male having a risk score less than 4 corresponds to a low chance of relapse; a risk score equal to 4 corresponds to a moderate chance of relapse; and a risk score greater than 4 corresponds to a high chance of relapse.

TABLE 21
Test validation estimates in Model 1 male.

| Risk score | | | | | |
|---|---|---|---|---|---|
| threshold | Sensitivity | Specificity | PPV | NPV | Sen + spec |
| 2 | 100% | 40% | 26% | 100% | 140% |
| 3 | 86% | 75% | 41% | 96% | 160% |
| 4 | 64% | 96% | 75% | 93% | 160% |
| 5 | 14% | 100% | 100% | 85% | 114% |
| 6 | 7% | 100% | 100% | 84% | 107% |

The risk scoring system using Model 2 to evaluate the 9 SNPs provided in Table 13 and 2 SNPs provided in Table 15 for females includes different levels of risk based on the subject's corresponding risk score. Referring to Table 22, in some embodiments, a female having a risk score less than 10 corresponds to a low chance of relapse; a risk score equal to 10 corresponds to a moderate chance of relapse; and a risk score greater than 10 corresponds to a high chance of relapse.

TABLE 22
Test validation estimates in Model 2 female.

| Risk score | | | | | |
|---|---|---|---|---|---|
| threshold | Sensitivity | Specificity | PPV | NPV | Sen + spec |
| 4 | 100% | 6% | 27% | 100% | 106% |
| 5 | 100% | 9% | 27% | 100% | 109% |
| 6 | 94% | 15% | 27% | 88% | 109% |
| 7 | 88% | 34% | 31% | 89% | 122% |
| 8 | 81% | 49% | 35% | 88% | 130% |
| 9 | 75% | 66% | 43% | 89% | 141% |
| 10 | 69% | 85% | 61% | 89% | 154% |
| 11 | 56% | 94% | 75% | 86% | 150% |
| 12 | 31% | 100% | 100% | 81% | 131% |
| 13 | 25% | 100% | 100% | 80% | 125% |
| 14 | 19% | 100% | 100% | 78% | 119% |
| 15 | 13% | 100% | 100% | 77% | 113% |

The risk scoring system using Model 2 to evaluate the 5 SNPs provided in Table 14 and 2 SNPs provided in Table 15 for males includes different levels of risk based on the subject's corresponding risk score. Referring to Table 23, in some embodiments, a male having a risk score less than 7 corresponds to a low chance of relapse; a risk score equal to 7 corresponds to a moderate chance of relapse; and a risk score greater than 7 corresponds to a high chance of relapse.

TABLE 23
Test validation estimates in Model 2 male.

| Risk score | | | | | |
|---|---|---|---|---|---|
| threshold | Sensitivity | Specificity | PPV | NPV | Sen + spec |
| 4 | 88% | 28% | 21% | 100% | 115% |
| 5 | 88% | 62% | 27% | 100% | 149% |
| 6 | 69% | 102% | 37% | 94% | 171% |
| 7 | 56% | 136% | 75% | 93% | 192% |
| 8 | 13% | 140% | 67% | 85% | 153% |
| 9 | 6% | 143% | 100% | 84% | 149% |

The risk scoring system using Model 3 to evaluate the 14 SNPs or 16 SNPs provided in Tables 13-15 for both females and males includes different levels of risk based on the subject's corresponding risk score. Referring to Table 24, in some embodiments, a subject having a risk score less than 5 corresponds to a low chance of relapse; a risk score equal to 5 corresponds to a moderate chance of relapse; and a risk score greater than 5 corresponds to a high chance of relapse.

TABLE 24
Test validation estimates in Model 3

| Risk score | | | | | |
|---|---|---|---|---|---|
| threshold | Sensitivity | Specificity | PPV | NPV | Sen + spec |
| 2 | 100% | 9% | 22% | 100% | 109% |
| 3 | 100% | 23% | 25% | 100% | 123% |
| 4 | 97% | 42% | 31% | 98% | 139% |
| 5 | 93% | 72% | 47% | 98% | 165% |
| 6 | 47% | 84% | 44% | 86% | 131% |
| 7 | 37% | 94% | 61% | 85% | 131% |
| 8 | 17% | 100% | 100% | 82% | 117% |
| 9 | 13% | 100% | 100% | 81% | 113% |
| 10 | 7% | 100% | 100% | 80% | 107% |

### Examples

### TaqMan SNP Genotyping

A SNP (single nucleotide polymorphism) is a change in the sequence of a gene at a specific locus. The sequence that matches the “normal” gene sequence is referred to as the wild-type allele, and the sequence that contains the change is referred to as the variant allele. A single gene may contain multiple SNPs that correspond with a functional alteration.

TaqMan SNP Genotyping Assays were obtained from Life Technologies. Each SNP assay contained primers and sequence-specific probes for identifying both the wild-type allele and the variant allele for a single SNP locus. The probes for the wild-type and variant alleles were tagged with different fluorophores. For example, an assay for a wild-type allele may contain a FAM probe and the corresponding variant allele assay may contain a VIC probe. Each probe emits a signal that is detectable at a different wavelength. The detector of the instrument measured the amount of each fluorescent signal in each reaction well. Gene sequence determinations were made based on the fluorescent signal as described below.

Genomic DNA (gDNA) contains two alleles, one inherited from each parent. Each allele pair is either the same (homozygous) or different (heterozygous). SNP genotyping data was performed by using these assays and analyzed using TaqMan Genotyper software provided amplification for only FAM (homozygous), only VIC (homozygous), or both FAM and VIC (heterozygous).

### Genomic DNA Isolation

Genomic DNA (gDNA) was isolated from the buccal swab samples using a Maxwell 16 LEV Blood DNA kit according to the manufacturer's suggested protocol (Promega). gDNA quality and concentration were measured using a NanoDrop 2000 spectrophotometer (Thermo Scientific).

### SNP Genotyping

SNPs were identified using Taqman qPCR chemistry, with assays run in an OpenArray format. A reaction mix, containing 50 ng gDNA (diluted with nuclease-free water) and Universal Master Mix II w/UNG were prepared. The reaction mix was then loaded into the OpenArray using an automated AccuFil instrument. The OpenArrays were run in a QuantStudio 12K Flex instrument (Life Technologies) using the following cycling parameters: 2 minutes @ 50° C.; 10 minutes @95° C.; and 50 cycles of 15 seconds at 92° C./90 seconds at 60° C.

### Control Samples

Positive control samples (gDNA samples from individuals with a confirmed genotype) were obtained for the Coriell Control Databank and positive control samples were included on each OpenArray.

### Data Analysis

Genetic test data generated included raw data files from 2 software programs—Genotyper and CopyCaller (Life Technologies). Each patient's data was analyzed, collated and assembled into a lab report template.

For some genes, there were multiple assays per gene. In order to produce a genotype determination, two separate companies were contracted: 1) Translational Software (TS, Seattle, Wash.)—analyzed the raw data files to produce a genotype call based on the individual assay data. and 2) Arivium, Inc. (Grand Rapids, Mich.)—to serve as the hosted LIMS system. Arivium developed a custom LIMS system that operates via a web-based portal to: a) transfer raw data, b) store reports.

Referring to Table 25, commonly used gene sequences and their corresponding SNPs for sixty (60) genes used to provide a risk score based upon the summing of counts is provided.

TABLE 25
Sequences

| | Gene | Genomic | |
|---|---|---|---|
| Seq No. | Symbol | Location | Sequence |
| 1 | ANKK1 | chr11: 113399438 | TCCCGTCAGGCTGACCCCAACCTGCA |
| | | | TGAGGCTGAGGGCAAGACCCCCCTC |
| 2 | ANKK1 | chr11: 113399438 | TCCCGTCAGGCTGACCCCAACCTGCG |
| | | | TGAGGCTGAGGGCAAGACCCCCCTC |
| 3 | BDNFOS/ | chr11: 27643996 | TACACAGGTGAATGAAAATGTCCACC |
| | antiBDNF | | GCTCTAGAAGAGTTTATACAAATAA |
| 4 | BDNFOS/ | chr11: 27643996 | TACACAGGTGAATGAAAATGTCCACT |
| | antiBDNF | | GCTCTAGAAGAGTTTATACAAATAA |
| 5 | CNIH3 | chr1: 224706393 | CAGGCAATGACGCACATAGCATCCTC |
| | | | GCCTGTTCCGGAGGGTCGCCTTTGA |
| 6 | CNIH3 | chr1: 224706393 | CAGGCAATGACGCACATAGCATCCTT |
| | | | GCCTGTTCCGGAGGGTCGCCTTTGA |
| 7 | CNR1 | chr6: 88150763 | TAGGTTTGTGGATGTGCCAGGACCAC |
| | | | GTAAGGAACAGCTCTCTCATATATT |
| 8 | CNR1 | chr6: 88150763 | TAGGTTTGTGGATGTGCCAGGACCAT |
| | | | GTAAGGAACAGCTCTCTCATATATT |
| 9 | CREBBP | chr16: 3745362 | TCCTTGCAATCAACGAAACTAGGAGA |
| | | | CAAAGAAGGCGCACTGTTAAAGCAC |
| 10 | CREBBP | chr16: 3745362 | TCCTTGCAATCAACGAAACTAGGAGG |
| | | | CAAAGAAGGCGCACTGTTAAAGCAC |
| 11 | CSNK1E | chr22: 38287631 | ACTAGGCCTCTCACACTGGATTCTGCA |
| | | | TTGGGGTGAACCACTTGCTACTCT |
| 12 | CSNK1E | chr22: 38287631 | ACTAGGCCTCTCACACTGGATTCTGG |
| | | | ATTGGGGTGAACCACTTGCTACTCT |
| 13 | DRD2 | chr11: 113425897 | CCAAAAATGTAGGGTATGGCAGTAAC |
| | | | GTTGAGGATAATTAAACTGCAGGGA |
| 14 | DRD2 | chr11: 113425897 | CCAAAAATGTAGGGTATGGCAGTAAT |
| | | | GTTGAGGATAATTAAACTGCAGGGA |
| 15 | DRD2 | chr11: 113441417 | GGTAGCCTATGGACCACATTTAGCTG |
| | | | GCATACAGGATTTGTTGGGCTCACA |
| 16 | DRD2 | chr11: 113441417 | GGTAGCCTATGGACCACATTTAGCTT |
| | | | GCATACAGGATTTGTTGGGCTCACA |
| 17 | DRD2 | chr11: 113426463 | AATTAAACTTATCAGCATTCCAAGGC |
| | | | GTTTCATACAAAGCACATGACTTCC |
| 18 | DRD2 | chr11: 113426463 | AATTAAACTTATCAGCATTCCAAGGT |
| | | | GTTTCATACAAAGCACATGACTTCC |
| 19 | DRD2 | chr11: 113414814 | AGGAAACAGGCTCATAGAAGGTAAG |
| | | | AAACTTGCCTAAGGTCACTCAGCAAA |
| 20 | DRD2 | chr11: 113414814 | AGGAAACAGGCTCATAGAAGGTAAGC |
| | | | AACTTGCCTAAGGTCACTCAGCAAA |
| 21 | DRD2 | chr11: 113412966 | CCCATCTCACTGGCCCCTCCCTTTCAC |
| | | | CCTCTGAAGACTCCTGCAAACACC |
| 22 | DRD2 | chr11: 113412966 | CCCATCTCACTGGCCCCTCCCTTTCCC |
| | | | CCTCTGAAGACTCCTGCAAACACC |
| 23 | DRD2/ | chr11: 113425564 | GAACCACATGATCAGATTCGCCTTTC |
| | Taq1B | | GAATAGGTGATTCTGACAGCACTGT |
| 24 | DRD2/ | chr11: 113425564 | GAACCACATGATCAGATTCGCCTTTTG |
| | Taq1B | | AATAGGTGATTCTGACAGCACTGT |
| 25 | DRD3 | chr3: 114162776 | ATAGGGAAGTGTTAGGTGAGGAGGGA |
| | | | TAGTTGTTGGAAAAGGGATGGAAGT |
| 26 | DRD3 | chr3: 114162776 | ATAGGGAAGTGTTAGGTGAGGAGGGG |
| | | | TAGTTGTTGGAAAAGGGATGGAAGT |
| 27 | DRD3 | chr3: 114140326 | AAAAGGCAGGTAATGATATTGTGACA |
| | | | TGGAGAATGTGCACTTAGAAGGGTC |
| 28 | DRD3 | chr3: 114140326 | AAAAGGCAGGTAATGATATTGTGACG |
| | | | TGGAGAATGTGCACTTAGAAGGGTC |
| 29 | DRD4 | chr11: 636784 | GGGCAGGGGGAGCGGGCGTGGAGGG |
| | | | CGCGCACGAGGTCGAGGCGAGTCCGC |
| 30 | DRD4 | chr11: 636784 | GGGCAGGGGGAGCGGGCGTGGAGGG |
| | | | TGCGCACGAGGTCGAGGCGAGTCCGC |
| 31 | GABRB3 | chr15: 26774621 | TCACGTTGGCATGTTTCTGTGCATTAA |
| | | | TTTTAAATATACTGCCTTTTTAAA |
| 32 | GABRB3 | chr15: 26774621 | TCACGTTGGCATGTTTCTGTGCATTGA |
| | | | TTTTAAATATACTGCCTTTTTAAA |
| 33 | GRIN3B | chr19: 1005231 | TTTATGTGGCCCCTGCACTGGTCCACG |
| | | | TGGCTGGGCGTCTTTGCGGCCCTG |
| 34 | GRIN3B | chr19: 1005231 | TTTATGTGGCCCCTGCACTGGTCCATG |
| | | | TGGCTGGGCGTCTTTGCGGCCCTG |
| 35 | intergenic | chr1: 163535374 | TTAGTAGACTTGAATTATAGATGCCA |
| | | | CAACTCTCATTCATGTGCATTTCTG |
| 36 | intergenic | chr1: 163535374 | TTAGTAGACTTGAATTATAGATGCCG |
| | | | CAACTCTCATTCATGTGCATTTCTG |
| 37 | OPRD1 | chr1: 28855013 | AAGGGTACAGCAGGGAACAAAATGG |
| | | | ACGAAGTCTTCTGGCTTCAGGGAACT |
| 38 | OPRD1 | chr1: 28855013 | AAGGGTACAGCAGGGAACAAAATGG |
| | | | GCGAAGTCTTCTGGCTTCAGGGAACT |
| 39 | OPRD1 | chr1: 28863085 | CGCTGCACCTGTGCATCGCGCTGGGC |
| | | | TACGCCAATAGCAGCCTCAACCCCG |
| 40 | OPRD1 | chr1: 28863085 | CGCTGCACCTGTGCATCGCGCTGGGTT |
| | | | ACGCCAATAGCAGCCTCAACCCCG |
| 41 | OPRM1 | chr6: 154040884 | TGGTGTTGATGTGTATATTCAAATACT |
| | | | ACATGTGAATGTGAAATGCCATAT |
| 42 | OPRM1 | chr6: 154040884 | TGGTGTTGATGTGTATATTCAAATATT |
| | | | ACATGTGAATGTGAAATGCCATAT |
| 43 | PENK | chr8: 56447926 | TGTATCCAATCCACCTATGCATCTACG |
| | | | TCTCCTAGACCTAGGGGGAAACCA |
| 44 | PENK | chr8: 56447926 | TGTATCCAATCCACCTATGCATCTATG |
| | | | TCTCCTAGACCTAGGGGGAAACCA |
| 45 | SLC6A3 | chr5: 1446274 | CAGCGCGCGGAGGAATGGAGCCCCCA |
| | | | GGCCGCCAAGGCCCAGGATGTCCAG |
| 46 | SLC6A3 | chr5: 1446274 | CAGCGCGCGGAGGAATGGAGCCCCCG |
| | | | GGCCGCCAAGGCCCAGGATGTCCAG |
| 47 | TACR1 | chr2: 75198602 | TACTGGCGAAGACAGCGGCGATGGGA |
| | | | AAGAAGTTGTGGAACTTGCAGTAGA |
| 48 | TACR1 | chr2: 75198602 | TACTGGCGAAGACAGCGGCGATGGGG |
| | | | AAGAAGTTGTGGAACTTGCAGTAGA |
| 49 | TACR1 | chr2: 75135918 | ACCTCCCCTATATTCTCCCCTCTCCAT |
| | | | TTCGCATTCTGTTTCACCATCGTT |
| 50 | TACR1 | chr2: 75135918 | ACCTCCCCTATATTCTCCCCTCTCCCT |
| | | | TTCGCATTCTGTTTCACCATCGTT |
| 51 | TACR3 | chr4: 103643921 | GATAACCCATAGAGAACCTTTTTCAA |
| | | | ATGATTGCCAAACACTGAAAGGCTT |
| 52 | TACR3 | chr4: 103643921 | GATAACCCATAGAGAACCTTTTTCAG |
| | | | ATGATTGCCAAACACTGAAAGGCTT |
| 53 | TACR3 | chr4: 103585232 | TAGTCAGTGTGGGTCCTGAGGTTGTA |
| | | | GCATGTTTAGCAAAGTTACAGAACA |
| 54 | TACR3 | chr4: 103585232 | TAGTCAGTGTGGGTCCTGAGGTTGTG |
| | | | GCATGTTTAGCAAAGTTACAGAACA |
| 55 | WLS | chr1: 68194522 | CTTCAAAACAATGTCACAAAAAATCA |
| | | | ACTGTGCTACAGTTCCCACCTGATT |
| 56 | WLS | chr1: 68194522 | CTTCAAAACAATGTCACAAAAAATCC |
| | | | ACTGTGCTACAGTTCCCACCTGATT |
| 57 | ZNF804A | chr2: 184668853 | ATTTATGAATTTAATTCATTAATGTCG |
| | | | TAAATAGTATTGCCCGAGAATTGG |
| 58 | ZNF804A | chr2: 184668853 | ATTTATGAATTTAATTCATTAATGTTG |
| | | | TAAATAGTATTGCCCGAGAATTGG |
| 59 | ZNF804A | chr2: 184913701 | AGATATCCAAGAAGTTGATTCTGATA |
| | | | GTTTTTGATTCTTTGTTTCAGTGTT |
| 60 | ZNF804A | chr2: 184913701 | AGATATCCAAGAAGTTGATTCTGATC |
| | | | GTTTTTGATTCTTTGTTTCAGTGTT |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

We claim:

1. A method for assessing whether a subject is at risk of opioid addiction, the method comprising:
(1) determining the presence of a plurality of pre-determined alleles in a biological sample from the subject by assigning a plurality of counts, wherein the plurality of pre-determined alleles comprise two or more genomic targets selected from Table 1;
(2) determining a risk score based upon summing the plurality of counts; and
(3) comparing the risk score with one or more predetermined reference values, wherein the subject is determined to have a risk level of opioid addiction if the risk score is greater than a threshold value using a SNP model.

2. The method of claim 1, wherein the SNP model comprises a sex-stratified single count SNP model, a sex-stratified double count SNP model, a non-sex-stratified single count SNP model, or any combinations thereof.

3. The method of either of claim 1 or 2, further comprising:
(4) administering a medical assisted treatment procedure to the subject based on the subject's risk score and risk level of opioid addiction.

4. The method of claim 3, wherein the medical assisted treatment procedure comprises monitoring the subject, prescribing an increase dose, prescribing a decreased dose, transitioning the subject from inpatient to outpatient rehabilitation, transitioning the subject from outpatient to inpatient rehabilitation, or any combinations thereof.

5. The method of any one of claims 1-4, wherein the plurality of pre-determined alleles comprise at least one allele selected from the group consisting of:
allele C+ of gene BDNFOS/antiBDNF (rs11030096);
allele A+ of gene DRD2 (rs1079596);
allele G+ of gene DRD2 (rs1125394);
allele C+ of gene DRD3 (rs9288993);
allele T/T of gene GABRB3 (rs4906902);
allele C/C of gene OPRM1 (rs510769);
allele T/T of gene TACR1 (rs735668);
allele T/T of gene ZNF804A (rs7597593);
allele C+ of gene DRD3 (rs2654754); and
allele A/A of gene OPRM1 (rs1799971).

6. The method of any one of claims 1-4, wherein the plurality of pre-determined alleles comprise at least one allele selected from the group consisting of:
allele A/A of gene CNR1 (rs2023239);
allele G+ of gene TACR3 (rs4530637);
allele C+ of gene TACR3 (rs1384401);
allele T/T of gene EXOC4 (rs718656);
allele T+ of gene DRD3 (rs324029);
allele G+ of gene DRD3 (rs6280);
allele G/G of gene CNR1 (rs6928499);
allele G/G of gene CYPB6 (rs3745274); and
allele C/C of gene CYP2D6 (rs1065852).

7. The method of any one of claims 1-4, wherein the plurality of pre-determined alleles comprise at least one allele selected from the group consisting of:
allele C/C of gene CNIH3 (rs1369846);
allele A/A of gene CNIH3 (rs1436171);
allele A/A of gene GRIN3A (rs17189632);
allele C+ of gene HTR3B (rs11606194);
allele C/C of gene OPRD1 (rs2234918);
allele G/G of gene WLS (rs1036066);
allele G+ of gene intergenic (rs965972);
allele C/C of gene MTHFR (rs1801133); and
allele G/G of gene MTHFR (rs1801133).

8. The method of any one of claims 1-4, wherein the plurality of pre-determined alleles comprise at least one allele selected from the group consisting of:
allele T/T of gene DRD3 (rs9825563);
allele T/T of gene GAL (rs948854);
allele C+ of gene NR4A2 (rs1405735);
allele A+ of gene OPRM (rs9479757); and
allele T+(A+) of gene CYP3A4 (rs35599367).

9. The method of any of claims 1-8, wherein the subject is a female.

10. The method of any of claims 1-8, wherein the subject is a male.

11. The method of any of claims 1-10, wherein the opioid addiction risk is opioid use disorder (OUD).

12. The method of any of claims 1-11, wherein the opioid addiction risk is a relapse risk.

13. A method of obtaining and utilizing an opioid use disorder (OUD) risk score for assessing a genetic predisposition to opioid addiction, the method comprising:
(1) obtaining a biological sample from a subject;
(2) performing an allelic analysis on the biological sample to determine the presence of a plurality of pre-determined alleles, wherein the plurality of pre-determined alleles comprise two or more genomic targets selected from Table 1;
(3) assigning a count for each of the alleles in the plurality of pre-determined alleles that was present in the biological sample;
(4) determining a risk score based upon a total count using a SNP Model, wherein the risk score identifies a severity of the genetic predisposition to opioid addiction; and
(5) administering a medical assisted treatment procedure based on the risk score identified in the subject.

14. The method of claim 13, wherein the SNP model comprises a sex-stratified single count SNP model, a sex-stratified double count SNP model, a non-sex-stratified single count SNP model, or any combinations thereof.

15. The method of either one of claim 13 or 14, wherein the medical assisted treatment procedure comprises monitoring the subject, prescribing an increase dose, prescribing a decreased dose, transitioning the subject from inpatient to outpatient rehabilitation, transitioning the subject from outpatient to inpatient rehabilitation, or any combinations thereof.

16. The method of any of claims 13-15, wherein the plurality of pre-determined alleles comprise at least one allele selected from the group consisting of:
allele C+ of gene BDNFOS/antiBDNF (rs11030096);
allele A+ of gene DRD2 (rs1079596);
allele G+ of gene DRD2 (rs1125394);
allele C+ of gene DRD3 (rs9288993);
allele T/T of gene GABRB3 (rs4906902);
allele C/C of gene OPRM1 (rs510769);
allele T/T of gene TACR1 (rs735668);
allele T/T of gene ZNF804A (rs7597593);
allele C+ of gene DRD3 (rs2654754); and
allele A/A of gene OPRM1 (rs1799971).

17. The method of any of claims 13-15, wherein the plurality of pre-determined alleles comprise at least one allele selected from the group consisting of:
allele A/A of gene CNR1 (rs2023239);
allele G+ of gene TACR3 (rs4530637);
allele C+ of gene TACR3 (rs1384401);
allele T/T of gene EXOC4 (rs718656);
allele T+ of gene DRD3 (rs324029);
allele G+ of gene DRD3 (rs6280);
allele G/G of gene CNR1 (rs6928499);
allele G/G of gene CYPB6 (rs3745274); and
allele C/C of gene CYP2D6 (rs1065852).

18. The method of any of claims 13-15, wherein the plurality of pre-determined alleles comprise at least one allele selected from the group consisting of:
allele C/C of gene CNIH3 (rs1369846);
allele A/A of gene CNIH3 (rs1436171);
allele A/A of gene GRIN3A (rs17189632);
allele C+ of gene HTR3B (rs11606194);
allele C/C of gene OPRD1 (rs2234918);
allele G/G of gene WLS (rs1036066);
allele G+ of gene intergenic (rs965972);
allele C/C of gene MTHFR (rs1801133); and
allele G/G of gene MTHFR (rs1801133).

19. The method of any of claims 13-15, wherein the plurality of pre-determined alleles comprise at least one allele selected from the group consisting of:
allele T/T of gene DRD3 (rs9825563);
allele T/T of gene GAL (rs948854);
allele C+ of gene NR4A2 (rs1405735);
allele A+ of gene OPRM (rs9479757); and
allele T+(A+) of gene CYP3A4 (rs35599367).

20. The method of any one of claims 13-19, wherein the subject is a female.

21. The method of either one of claims 13-19, wherein the subject is a male.

22. The method of any of claims 13-21, wherein the opioid addiction risk is opioid use disorder (OUD).

23. The method of any of claims 13-22, wherein the opioid addiction risk is relapse risk.

24. A method of obtaining and utilizing a relapse risk score for assessing a genetic predisposition to addiction relapse, the method comprising:
(1) obtaining a biological sample from a subject;
(2) performing an allelic analysis on the biological sample to determine the presence of a plurality of pre-determined alleles, wherein the plurality of pre-determined alleles comprise two or more genomic targets selected from Table 1;
(3) assigning a count for each of the alleles in the plurality of pre-determined alleles that was present in the biological sample;
(4) determining a risk score based upon a total count using a SNP Model, wherein the risk score identifies a severity of the genetic predisposition to opioid addiction; and
(5) administering a medical assisted treatment procedure based on the risk score identified in the subject.

25. The method of claim 24, wherein the SNP model comprises a sex-stratified single count SNP model, a sex-stratified double count SNP model, a non-sex-stratified single count SNP model, or any combinations thereof.

26. The method of either one of claim 24 or 25, wherein the medical assisted treatment procedure comprises monitoring the subject, prescribing an increase dose, prescribing a decreased dose, transitioning the subject from inpatient to outpatient rehabilitation, transitioning the subject from outpatient to inpatient rehabilitation, or any combinations thereof.

27. The method of any of claims 24-26, wherein the plurality of pre-determined alleles comprise at least one allele selected from the group consisting of:
allele C+ of gene BDNFOS/antiBDNF (rs11030096);
allele A+ of gene DRD2 (rs1079596);
allele G+ of gene DRD2 (rs1125394);
allele C+ of gene DRD3 (rs9288993);
allele T/T of gene GABRB3 (rs4906902);
allele C/C of gene OPRM1 (rs510769);
allele T/T of gene TACR1 (rs735668);
allele T/T of gene ZNF804A (rs7597593);
allele C+ of gene DRD3 (rs2654754); and
allele A/A of gene OPRM1 (rs1799971).

28. The method of any of claims 24-26, wherein the plurality of pre-determined alleles comprise at least one allele selected from the group consisting of:
allele A/A of gene CNR1 (rs2023239);
allele G+ of gene TACR3 (rs4530637);
allele C+ of gene TACR3 (rs1384401);
allele T/T of gene EXOC4 (rs718656);
allele T+ of gene DRD3 (rs324029);
allele G+ of gene DRD3 (rs6280);
allele G/G of gene CNR1 (rs6928499);
allele G/G of gene CYPB6 (rs3745274); and
allele C/C of gene CYP2D6 (rs1065852).

29. The method of any of claims 24-26, wherein the plurality of pre-determined alleles comprise at least one allele selected from the group consisting of:
allele C/C of gene CNIH3 (rs1369846);
allele A/A of gene CNIH3 (rs1436171);
allele A/A of gene GRIN3A (rs17189632);
allele C+ of gene HTR3B (rs11606194);
allele C/C of gene OPRD1 (rs2234918);
allele G/G of gene WLS (rs1036066);
allele G+ of gene intergenic (rs965972);
allele C/C of gene MTHFR (rs1801133); and
allele G/G of gene MTHFR (rs1801133).

30. The method of any of claims 24-26, wherein the plurality of pre-determined alleles comprise at least one allele selected from the group consisting of:
allele T/T of gene DRD3 (rs9825563);
allele T/T of gene GAL (rs948854);
allele C+ of gene NR4A2 (rs1405735);
allele A+ of gene OPRM (rs9479757); and
allele T+(A+) of gene CYP3A4 (rs35599367).

31. The method of any one of claims 24-30, wherein the subject is a female.

32. The method of any one of claims 24-30, wherein the subject is a male.

33. The method of any of claims 24-32, wherein the addiction relapse is an opioid use disorder (OUD) or opioid addition relapse.

34. A method for assessing whether a subject is at risk of opioid addiction, the method comprising:
(1) determining the presence of a plurality of pre-determined alleles in a biological sample from the subject by assigning a plurality of counts, wherein the plurality of pre-determined alleles comprises two or more genomic targets selected in Table 1;
(2) determining a risk score based upon summing the plurality of counts;
(3) comparing the risk score with a predetermined reference value using a SNP Model, wherein the subject is determined to be at high risk of opioid addiction if the risk score is greater than a threshold value as compared to those subjects where the risk score is lower than the threshold value; and
(4) administering a medical assisted treatment procedure based on the risk score identified in the subject.

35. The method of claim 34, wherein the SNP model comprises a sex-stratified single count SNP model, a sex-stratified double count SNP model, a non-sex-stratified single count SNP model, or any combinations thereof.

36. The method of either one or claim 34 or 35, wherein the medical assisted treatment procedure comprises monitoring the subject, prescribing an increase dose, prescribing a decreased dose, transitioning the subject from inpatient to outpatient rehabilitation, transitioning the subject from outpatient to inpatient rehabilitation, or any combinations thereof.

37. The method of any of claims 34-36, wherein the plurality of pre-determined alleles comprise at least one allele selected from the group consisting of:
allele C+ of gene BDNFOS/antiBDNF (rs11030096);
allele A+ of gene DRD2 (rs1079596);
allele G+ of gene DRD2 (rs1125394);
allele C+ of gene DRD3 (rs9288993);
allele T/T of gene GABRB3 (rs4906902);
allele C/C of gene OPRM1 (rs510769);
allele T/T of gene TACR1 (rs735668);
allele T/T of gene ZNF804A (rs7597593);
allele C+ of gene DRD3 (rs2654754); and
allele A/A of gene OPRM1 (rs1799971).

38. The method of any of claims 34-36, wherein the plurality of pre-determined alleles comprise at least one allele selected from the group consisting of:
allele A/A of gene CNR1 (rs2023239);
allele G+ of gene TACR3 (rs4530637);
allele C+ of gene TACR3 (rs1384401);
allele T/T of gene EXOC4 (rs718656);
allele T+ of gene DRD3 (rs324029);
allele G+ of gene DRD3 (rs6280);
allele G/G of gene CNR1 (rs6928499);
allele G/G of gene CYPB6 (rs3745274); and
allele C/C of gene CYP2D6 (rs1065852).

39. The method of any of claims 34-36, wherein the plurality of pre-determined alleles comprise at least one allele selected from the group consisting of:
allele C/C of gene CNIH3 (rs1369846);
allele A/A of gene CNIH3 (rs1436171);
allele A/A of gene GRIN3A (rs17189632);
allele C+ of gene HTR3B (rs11606194);
allele C/C of gene OPRD1 (rs2234918);
allele G/G of gene WLS (rs1036066);
allele G+ of gene intergenic (rs965972);
allele C/C of gene MTHFR (rs1801133); and
allele G/G of gene MTHFR (rs1801133).

40. The method of any of claims 34-36, wherein the plurality of pre-determined alleles comprise at least one allele selected from the group consisting of:
allele T/T of gene DRD3 (rs9825563);
allele T/T of gene GAL (rs948854);
allele C+ of gene NR4A2 (rs1405735);
allele A+ of gene OPRM (rs9479757); and
allele T+(A+) of gene CYP3A4 (rs35599367).

41. The method of any one of claims 34-36, wherein the plurality of pre-determined alleles comprise at least one allele selected from the group consisting of:
chr11:113399438 of gene ANKK1;
chr11:27643996 of gene BDNFOS/antiBDNF;
chr1:224706393 of gene CNIH3;
chr6:88150763 of gene CNR1;
chr16:3745362 of gene CREBBP;
chr22:38287631 of gene CSNK1E;
chr11:113425897 of gene DRD2;
chr11:113441417 of gene DRD2;
chr11:113426463 of gene DRD2;
chr11:113414814 of gene DRD2;
chr11:113412966 of gene DRD2;
chr11:113425564 of gene DRD2;
chr3:114162776 of gene DRD3;
chr3:114140326 of gene DRD3;
chr11:636784 of gene DRD4;
chr15:26774621 of gene GABRB3;
chr19:1005231 of gene GABRB3;
chr1:163535374 of gene intergenic g.163535374G;
chr1:28855013 of gene OPRD1;
chr1:28863085 of gene OPRD1;
chr6:154040884 of gene OPRM1;
chr8:56447926 of gene PENK;
chr5:1446274 of gene SLC6A3;
chr2:75198602 of gene TACR1;
chr2:75135918 of gene TACR1;
chr4:103643921 of gene TACR3;
chr4:103585232 of gene TACR3;
chr1:68194522 of gene WLS;
chr2:184668853 of gene ZNF804A; and
chr2:184913701 of gene ZNF804A.

42. The method of claim 34, wherein the plurality of pre-determined alleles further comprise at least one allele selected from the group consisting of:
chr6:154039662 of gene OPRM1 118A>G;
chr19:41006936 of gene CYP2B6*13*6*7*9+516G>T;
chr22:42130692 of gene CYP2D6*4*10*1 4A+100C>T;
chr1:11796321 of gene MTHFR 677C>T;
CYP2C9 non EM (IM or PM); and
chr7:99768693 of gene CYP3A4*22 intron6 15389C>T.

43. The method of any of claims 34-42, wherein the opioid addiction risk is opioid use disorder (OUD).

44. The method of any of claims 34-43, wherein the opioid addiction risk is relapse risk.

45. The method of any of claims 34-44, wherein the subject is a female.

46. The method of any of claims 34-44, wherein the subject is a male.

47. A method of obtaining and utilizing a relapse risk score for assessing a genetic predisposition to addiction relapse, the method comprising:
(1) obtaining a biological sample from a subject;
(2) performing an allelic analysis on the biological sample to determine the presence of a plurality of pre-determined alleles by assigning a plurality of counts, wherein the plurality of pre-determined alleles comprise one or more genomic targets selected from Table 1;
(3) determining a risk score based upon summing the plurality of counts; and
(4) comparing the risk score with one or more predetermined reference values, wherein the subject is determined to have a risk level of opioid addiction if the risk score is greater than a threshold value using a SNP model.

48. The method of claim 47, wherein the SNP model comprises a sex-stratified single count SNP model, a sex-stratified double count SNP model, a non-sex-stratified single count SNP model, or any combinations thereof.

49. The method of either of claim 47 or 48, further comprising:
(5) administering a medical assisted treatment procedure to the subject based on the subject's risk score and risk level of opioid addiction.

50. The method of claim 49, wherein the medical assisted treatment procedure comprises monitoring the subject, prescribing an increase dose, prescribing a decreased dose, transitioning the subject from inpatient to outpatient rehabilitation, transitioning the subject from outpatient to inpatient rehabilitation, or any combinations thereof.

51. The method of any one of claims 47-50, wherein the plurality of pre-determined alleles comprise at least one allele selected from the group consisting of:
allele C+ of gene BDNFOS/antiBDNF (rs11030096);
allele A+ of gene DRD2 (rs1079596);
allele G+ of gene DRD2 (rs1125394);
allele C+ of gene DRD3 (rs9288993);
allele T/T of gene GABRB3 (rs4906902);
allele C/C of gene OPRM1 (rs510769);
allele T/T of gene TACR1 (rs735668);
allele T/T of gene ZNF804A (rs7597593);
allele C+ of gene DRD3 (rs2654754); and
allele A/A of gene OPRM1 (rs1799971).

52. The method of any one of claims 47-50, wherein the plurality of pre-determined alleles comprise at least one allele selected from the group consisting of:
allele A/A of gene CNR1 (rs2023239);
allele G+ of gene TACR3 (rs4530637);
allele C+ of gene TACR3 (rs1384401);
allele T/T of gene EXOC4 (rs718656);
allele T+ of gene DRD3 (rs324029);
allele G+ of gene DRD3 (rs6280);
allele G/G of gene CNR1 (rs6928499);
allele G/G of gene CYPB6 (rs3745274); and
allele C/C of gene CYP2D6 (rs1065852).

53. The method of any one of claims 47-50, wherein the plurality of pre-determined alleles comprise at least one allele selected from the group consisting of:
allele C/C of gene CNIH3 (rs1369846);
allele A/A of gene CNIH3 (rs1436171);
allele A/A of gene GRIN3A (rs17189632);
allele C+ of gene HTR3B (rs11606194);
allele C/C of gene OPRD1 (rs2234918);
allele G/G of gene WLS (rs1036066);
allele G+ of gene intergenic (rs965972);
allele C/C of gene MTHFR (rs1801133); and
allele G/G of gene MTHFR (rs1801133).

54. The method of any one of claims 47-50, wherein the plurality of pre-determined alleles comprise at least one allele selected from the group consisting of:
allele T/T of gene DRD3 (rs9825563);
allele T/T of gene GAL (rs948854);
allele C+ of gene NR4A2 (rs1405735);
allele A+ of gene OPRM (rs9479757); and
allele T+(A+) of gene CYP3A4 (rs35599367).

55. The method of any of claims 47-54, wherein the subject is a female.

56. The method of any of claims 47-54, wherein the subject is a male.

57. The method of any of claims 47-56, wherein the opioid addiction risk is opioid use disorder (OUD).

58. The method of any of claims 47-57, wherein the opioid addiction risk is a relapse risk.
